(19) United States
(12) Patent Application Publication
(10) Pub. No.: US 2025/0188513 A1
(43) Pub. Date: Jun. 12, 2025

(54) METHODS OF BARCODING NUCLEIC ACIDS FOR DETECTION AND SEQUENCING

(71) Applicant: UNIVERSAL SEQUENCING TECHNOLOGY CORPORATION, Canton, MA (US)

(72) Inventors: Zhoutao Chen, Carlsbad, CA (US); Ivan G. Bassets, La Jolla, CA (US)

(73) Assignee: UNIVERSAL SEQUENCING TECHNOLOGY CORPORATION, Canton, MA (US)

(21) Appl. No.: 19/058,949

(22) Filed: Feb. 20, 2025

Related U.S. Application Data
(63) Continuation of application No. PCT/US2023/073042, filed on Aug. 29, 2023.
(60) Provisional application No. 63/373,778, filed on Aug. 29, 2022.

(51) Int. Cl. C12Q 1/6806; C12Q 1/6844; C12Q 1/6869; C12Q 1/6876

(52) U.S. Cl. CPC C12Q 1/6806; C12Q 1/6844; C12Q 1/6869; C12Q 1/6876

(57) ABSTRACT

Described and featured herein are methods to barcode nucleic acids for detection and sequencing, particularly at a single cell level. The methods involve application of a barcode template in a compartment with various targets, including nucleic acid fragments, nuclei and/or cells. After clonal amplification within the compartment, the barcode sequence integrates into its target before the compartment is broken so that it will effectively barcode nucleic acid fragments originated from a nucleic acid fragment, a nucleus or a cell clonally. The barcode information can be used for tracking the origin of the fragment, nucleus or cell and be used for haplotype phasing and a variety of single cell-based applications including whole genome sequencing, metagenome sequencing, targeted sequencing, RNA sequencing and immune repertoire sequencing.

### CROSS REFERENCE TO RELATED APPLICATIONS

This application is a continuation under 35 U.S.C. § 111(a) of PCT International Patent Application No. PCT/US2023/073042, filed Aug. 29, 2023, designating the United States and published in English, which claims priority to and the benefit of U.S. Provisional Application No. 63/373,778, filed Aug. 29, 2022, the entire contents of each of which are incorporated by reference herein.

### FIELD

The present disclosure is in the technical field of genomics. More particularly, the disclosure relates generally to nucleic acid sequencing. More specifically, the disclosure relates to methods for improved nucleic acid detection and sequencing for single cell analysis, haplotype phasing, de novo assembly and variant detection.

### BACKGROUND

Nucleic acid sequencing can provide information for a wide variety of biomedical applications, including diagnostics, prognostics, pharmacogenomics, and forensic biology. Sequencing may involve basic low-throughput methods including Maxam-Gilbert sequencing (chemically modified nucleotide) and Sanger sequencing (chain-termination) methods, or high-throughput, next-generation methods including massively parallel pyrosequencing, sequencing by synthesis, sequencing by ligation, semiconductor sequencing, and others. For most sequencing methods, a sample, such as a nucleic acid target, needs to be processed prior to introduction into a sequencing instrument. For example, a sample may be fragmented, amplified or attached to an identifier. Unique identifiers are often used to identify the origin of a target. Most sequencing methods generate relatively short sequencing reads, ranging from tens of bases to hundreds of bases in length, and cannot generate complete haplotype phase information due to limited sequencing read length. Most biological samples contain many cells. And most assays measure responses for bulk cells, not at an individual cell level. Needed in the art are new methods for genotyping cells at the single cell level, for example, in order to separate tumor cells from wild type or normal cells in a sample. Such methods are provided by the methods and the features thereof as described herein.

### SUMMARY

The present disclosure provides methods for improved nucleic acid detection and sequencing. In particular, the present disclosure provides improved methods for single cell nucleic acid sequencing and detection.

In an aspect, the present disclosure provides a method of single cell sequencing to characterize a biological sample at an individual cell level. The method involves sequestering a plurality of cells or a plurality of nuclei into compartments, where each cell or nucleus is sequestered into a separate compartment with a plurality of barcode templates, where each barcode template includes a barcode sequence, and where at least some compartments include more than one population of barcode templates, each population of barcode templates having a unique barcode sequence different from that of other populations of barcode templates. The method involves amplifying at least one type of cellular content in each cell or nucleus into a plurality of copies and fragmenting the cellular content in each compartment into a plurality of fragments. The method involves attaching a barcode template to each fragment. The method involves collecting the barcode template attached fragments. The method involves sequencing the barcode attached fragments and classifying fragments with a same barcode sequence as belonging to a same cellular unit.

In an aspect, the present disclosure provides a method of single cell sequencing to characterize a biological sample at an individual cell level. The method involves sequestering a plurality of cells or a plurality of nuclei and a plurality of barcode templates into compartments, where each cell or nucleus is sequestered into a separate compartment with at least one barcode template including a barcode sequence, and wherein at least some compartments include at least two different barcode templates, each different barcode template having a different barcode sequence. The method involves amplifying at least one type of cellular content in each cell or nucleus into a plurality of copies and fragmenting the cellular content in each compartment into fragments, and amplifying the at least one barcode template in each compartment. The method involves attaching a barcode template to each fragment. The method involves collecting the barcode template attached fragments. The method involves sequencing the barcode attached fragments and classifying fragments with a same barcode sequence as belonging to a same cellular unit.

In an aspect, the present disclosure provides a method of single cell transcriptome sequencing. The method involves generating cDNA from cellular or nuclear RNA of a cell or nucleus in a plurality of cells or nuclei. The method involves tagmenting the generated cDNA randomly across an entire length of the cDNA in each of the cells or nuclei using a plurality of transpososomes, to form a plurality of tagmented cDNA fragments, where each transpososome includes at least one transposon and one transposase. The method involves sequestering the plurality of cells or nuclei into compartments, where each cell or nucleus is sequestered into a separate compartment with a plurality of barcode templates, where each barcode template includes a barcode sequence. The method involves attaching a barcode template to each tagmented cDNA fragment in the compartment. The method involves collecting the barcode attached cDNA fragments. The method involves sequencing the barcode and barcode attached cDNA fragments to characterize a transcriptome profile of each cell or nucleus on a single cell basis.

In an aspect, the present disclosure provides a method of single cell transcriptome sequencing. The method involves generating cDNA from cellular or nuclear RNA from a cell or nucleus in a plurality of cells or nuclei. The method involves tagmenting the generated cDNA randomly across an entire length of the cDNA in each of the cells or nuclei using a plurality of transpososomes, to form a plurality of tagmented cDNA fragments, where each transpososome includes at least one transposon and one transposase. The method involves sequestering the cells or nuclei and a plurality of barcode templates, where each cell or nucleus is sequestered into a separate compartment with at least one barcode template. The method involves attaching a barcode template to each tagmented cDNA fragment. The method involves collecting the barcode attached cDNA fragments. The method involves sequencing the barcode and barcode attached cDNA fragments to characterize the transcriptome profile of each cell on a single cell basis.

In any of the above aspects, or embodiments thereof, each barcode template is a nucleotide sequence, capable of functioning as a unique identifier.

In any of the above aspects, or embodiments thereof, each barcode template exists freely in solution. In any of the above aspects, or embodiments thereof, each barcode template is immobilized on a carrier. In any of the above aspects, or embodiments thereof, the carrier is a solid bead or particle, a dissolvable bead or particle, or a combination thereof.

In any of the above aspects, or embodiments thereof, the type of cellular content is RNA, DNA, RNA/DNA hybrid, protein, metabolite, ligand, chemical compound, drug, macromolecule, or a combination thereof. In any of the above aspects, or embodiments thereof, the type of cellular content is RNA, DNA, an RNA/DNA hybrid, or a combination thereof.

In any of the above aspects, or embodiments thereof, the fragment is directly attached to the barcode template. In any of the above aspects, or embodiments thereof, the fragment is indirectly attached to the barcode template. In any of the above aspects, or embodiments thereof, the fragment is attached to a linker oligo, or an adapter, where the linker oligo or the adapter is attached to the barcode template.

In any of the above aspects, or embodiments thereof, the cellular content is endogenous. In any of the above aspects, or embodiments thereof, the cellular content is exogenous.

In any of the above aspects, or embodiments thereof, the compartment includes a cell or a nucleus without further compartmentation; a tube or microtube; a well or microwell; a plate; a well in a multi-well plate; a slide; a spot on a slide; a droplet; a tubing; a channel; a bottle; a chamber; or a flow-cell.

In any of the above aspects, or embodiments thereof, the amplifying the cellular content and/or barcode template step and the attaching the barcode template to the fragments step occur substantially simultaneously.

In any of the above aspects, or embodiments thereof, the method also involves identifying barcode sequences attached to cellular content originating from the same cell or nucleus, and merging cellular units corresponding to barcode sequences identified as attached to cellular content originating from the same cell or nucleus.

In any of the above aspects, or embodiments thereof, the cells are eukaryotic, prokaryotic, or a combination thereof.

In any of the above aspects, or embodiments thereof, the plurality of barcode templates in each compartment includes at least two populations of barcode templates, where each population of barcode templates has a different barcode sequence.

In any of the above aspects, or embodiments thereof, the attaching results in at least two populations of cDNA fragments each attached to a different population of barcode templates.

In any of the above aspects, or embodiments thereof, the at least one barcode template is at least two different barcode templates, each having a different barcode sequence.

In any of the above aspects, or embodiments thereof, the generated cDNA is first strand cDNA and forms a DNA/RNA hybrid with the cellular or nuclear RNA.

In any of the above aspects, or embodiments thereof, the generated cDNA is first and second stranded cDNA, and forms double stranded DNA.

In any of the above aspects, or embodiments thereof, the generated cDNA includes transcripts including both the 3′ end and the 5′ end of the cellular or nuclear RNA.

In any of the above aspects, or embodiments thereof, the transcriptome profile includes both a 3′ end and a 5′ end of the cellular or nuclear RNA.

In any of the above aspects, or embodiments thereof, the sequences of the barcode template attached cDNA fragments are converted into full length RNA sequences.

In any of the above aspects, or embodiments thereof, the attaching the barcode template to the tagmented cDNA fragment includes amplifying the barcode templates and/or amplifying the tagmented cDNA fragments.

In any of the above aspects, or embodiments thereof, the amplifying the barcode templates and the amplifying the tagmented cDNA fragments occurs separately.

In any of the above aspects, or embodiments thereof, amplifying the barcode templates and the amplifying the tagmented cDNA fragments occurs simultaneously.

In any of the above aspects, or embodiments thereof, the at least one barcode template in each compartment is a single barcode template

In any of the above aspects, or embodiments thereof, the plurality of barcode templates in each compartment is a plurality of copies of a same barcode template.

In any of the above aspects, or embodiments thereof, the cell or nucleus, or the plurality of cells or nuclei, is obtained from a biological sample or cell culture.

### Further Aspects

In an aspect, methods of amplifiable single cell sequencing to characterize a biological sample at individual cell level are described and provided. The methods include providing a plurality of cells or nuclei from a sample, providing a plurality of barcode templates, sequestering a cell or a cell nucleus with more than one different barcode template in one compartment; amplifying each barcode template into a plurality of copies, and amplifying one type or more than one type of cellular content into a plurality of copies, wherein the cellular content comprises nucleic acid sequences naturally or attached to a nucleic acid sequence artificially, in the sequestered compartment; coupling an amplified barcode template with an amplified cellular content in the compartment; sequencing to determine the barcode sequence in the barcode template and its associated cellular content sequence; classifying the cellular content with the same barcode sequence as one cellular unit or part of a cellular unit. In an embodiment, the amplification step and coupling step can occur sequentially or simultaneously. These methods make one cellular content become more than one cellular unit. In embodiments, the cellular content comprises DNA, RNA, protein, lipid, or an organelle within a cell internally, or a nucleus, or associated with a cell externally, or a combination thereof. In embodiments, the cell is a eukaryotic and/or a prokaryotic cell. In embodiments, the compartment is a well, microwell, droplet, microdroplet, hole and other material which is capable of physically sequestering the cellular content into different reaction units or spaces.

In an aspect, a method of sequencing a single-cell, full-length transcriptome is provided, in which the method comprises providing a plurality of cells from a biological sample; contacting the cells with a reverse transcriptase and a primer, e.g., an oligo-dT primer, to generate a first strand cDNA in situ; providing a plurality of transpososomes, each transpososome comprising at least one transposon and one transposase; tagmenting the RNA/cDNA hybrid transcripts randomly across the entire transcripts in situ; providing a plurality of barcode templates and amplification reagent; compartmentalizing the cells, the barcode templates, and amplification reagents to generate two or more compartments, wherein each compartment comprises a cell, one or more than one barcode templates with different barcode sequences, and amplification reagent; amplifying the barcode template and tagmented RNA/cDNA fragments, attaching said barcode sequence to cDNA fragments or fragments generated from cDNA so that a plurality of barcode attached fragments sharing the same one or more barcode sequences are presented in the compartment; collecting the barcode attached fragments; sequencing the barcode and barcoded tagged nucleic acid to characterize the full-length transcriptome profile on a single cell basis.

In another aspect, methods of tracking a target's origin by barcode tagging are provided. The methods include encapsulating at least one unique barcode template with at least one target in a compartment; amplifying the barcode template(s) and modifying the target, wherein the modified target is capable of linking to a barcode in the compartment; linking a barcode sequence to a modified target so that a plurality of modified targets sharing the same one or more barcode sequences are presented in the compartment; removing the compartments and collecting the barcode tagged modified targets for downstream applications. In an embodiment, a target is selected from a group consisting of a nucleic acid, a protein including antibody, a ligand, a chemical compound, a nucleus, a cell, and a combination thereof. In an embodiment, a cell can be prokaryotic or eukaryotic. In an embodiment, the modification for a target is selected from the group consisting of strand transfer reaction, tagmentation reaction, reverse transcription, amplification, primer extension, restriction digestion, hybridization, ligation, fragmentation, and a combination thereof. In some embodiments, a target is treated and/or modified before encapsulation. A treatment is selected from the group consisting of denaturation, permeabilization, fixation, labeling, conjugation, in situ reactions, and a combination thereof. In some embodiments, the compartment origin of different barcode sequences presented in the same compartment can be identified based on their shared compartment content.

In some embodiments, a barcode template comprises a central barcode sequence flanked by at least two handle sequences which can be used as priming sites, hybridization sites or binding sites.

In another aspect, methods of tracking nucleic acid fragment origin by barcode tagging is provided. The methods include providing a plurality of nucleic acid targets and a plurality of transpososomes, each transpososome comprises at least one transposon and one transposase; incubating nucleic acid targets and transpososomes together to form strand transfer complexes (STCs) on the nucleic acid targets; providing a plurality of unique barcode templates; compartmentalizing the nucleic acid targets with STCs and the barcode templates to generate two or more compartments comprising both one or more nucleic acid targets and one or more than one barcode templates with different barcode sequences; amplifying the barcode template in the compartment, fragmenting nucleic acid target by breaking the STCs to form tagmented nucleic acid fragments, and attaching barcode sequence to tagmented nucleic acid fragments so that a plurality of fragments sharing the same one or more barcode sequences are presented in the compartment; removing the compartments and collecting the barcode tagged nucleic acid fragments.

In another aspect, methods of tracking nucleic acid fragment origin by barcode tagging are provided. The methods include providing a plurality of nucleic acid targets and a plurality of transpososomes, each transpososome comprising at least one transposon and one transposase; incubating nucleic acid targets and transpososomes together to form strand transfer complexes (STCs) on the nucleic acid targets; providing a plurality of unique barcode templates; compartmentalizing the nucleic acid targets with STCs and the barcode templates to generate two or more compartments comprising both one or more nucleic acid targets and one or more than one barcode templates with different barcode sequences; attaching a barcode sequence to the nucleic acid targets in the compartment by i) fragmenting the nucleic acid target by breaking the STCs to form tagmented nucleic acid fragments; ii) amplifying the tagmented nucleic acid fragments with non-target-specific primers (i.e. only transposon specific), and amplifying the barcode template(s); iii) linking a barcode template to a tagmented nucleic acid fragment, wherein a plurality of fragments sharing the same one or more barcode sequences are presented in the compartment; removing the compartments and collecting the barcode tagged nucleic acid fragments for downstream applications. By way of example, a downstream application comprises generating haplotype phased sequencing information.

In another aspect, methods of tracking targeted nucleic acid fragment origin by barcode tagging are provided. The methods include providing a plurality of nucleic acid targets, a plurality of target specific primers and a plurality of transpososomes, wherein each transpososome comprises at least one transposon and one transposase; incubating nucleic acid targets and transpososomes together to form strand transfer complexes (STCs) on the nucleic acid targets; providing a plurality of unique barcode templates; compartmentalizing the nucleic acid targets with STCs and the barcode templates to generate two or more compartments comprising one or more nucleic acid targets and one or more than one barcode template with different barcode sequences; attaching a barcode sequence to the nucleic acid targets in the compartment by i) fragmenting the nucleic acid target by breaking the STCs to form tagmented nucleic acid fragments; ii) amplifying the tagmented nucleic acid fragments with a transposon specific primer and a target-specific primer, and amplifying the barcode template(s); iii) linking a barcode template to a tagmented nucleic acid fragment, wherein a plurality of fragments sharing the same one or more barcode sequences are presented in the compartment; removing the compartments; and collecting the barcode tagged nucleic acid fragments. In some embodiments, the nucleic acid targets are within a cell or nucleus, wherein the cells or the nuclei are permeabilized or fixed, and then incubated with a plurality of transpososomes before being compartmentalized with target specific primers and barcode templates.

In another aspect, methods of tracking targeted nucleic acid fragment origin by barcode tagging are provided. The methods include providing a plurality of nucleic acid fragments, a plurality of unique barcode templates and a plurality of target specific primers wherein at least some the target specific primers are capable of attaching to barcode templates directly or indirectly; compartmentalizing the nucleic acid fragments, target specific primers and the barcode templates to generate two or more compartments comprising one or more nucleic acid fragments, target specific primers and one or more than one barcode template with different barcode sequences; attaching a barcode sequence to the nucleic acid fragments in the compartment by i) amplifying the targets from the nucleic acid fragments using target-specific primers, and amplifying the barcode template(s); ii) linking a barcode template to an amplified nucleic acid target in the compartment, wherein a plurality of amplified nucleic acid targets sharing the same one or more barcode sequences are presented in the compartment; iii) removing the compartments and iv) collecting the barcoded nucleic acid targets for further analyses, for example, sequencing.

In one aspect, methods of single cell ATAC-seq are provided. The methods include providing a plurality of cells or nuclei and a plurality of transpososomes, wherein each transpososome comprises at least one transposon and one transposase; incubating the plurality of cells or nuclei and the plurality of transpososomes together to form strand transfer complexes (STCs) on accessible chromatin in the cells or nuclei; providing a plurality of unique barcode templates; compartmentalizing the treated cells or nuclei and barcode templates to generate two or more compartments comprising both a cell or a nucleus and one or more than one barcode template with different barcode sequences; amplifying the barcode template in the compartment, breaking cellular and/or nuclear membrane, fragmenting accessible chromatin by breaking the STCs to form tagmented nucleic acid fragments, and attaching a barcode sequence to tagmented nucleic acid fragments so that a plurality of fragments sharing the same one or more barcode sequences are presented in the compartment; removing the compartments and collecting the barcode tagged nucleic acid fragments; sequencing the barcode and barcode tagged nucleic acid to characterize the accessible chromatin region on a single cell basis.

In one aspect, methods of single cell ATAC-seq are provided, which include providing a plurality of cells or nuclei and a plurality of transpososomes, wherein each transpososome comprises at least one transposon and one transposase; incubating the plurality of cells or nuclei and the plurality of transpososomes together to form strand transfer complexes (STCs) on accessible chromatin in the nuclei; providing a plurality of unique barcode templates; compartmentalizing the treated cells or nuclei and barcode templates to generate two or more compartments comprising both a cell or a nucleus and one or more than one barcode templates with different barcode sequences; attaching a barcode sequence to accessible chromatin fragments in the compartment by i) breaking the cellular and/or nuclear membrane, and fragmenting accessible chromatin by breaking the STCs to form tagmented nucleic acid fragments; ii) amplifying the tagmented nucleic acid fragments and amplifying the barcode template; iii) linking a barcode template to a tagmented nucleic acid fragment, wherein a plurality of fragments sharing the same one or more barcode sequences are presented in the compartment; removing the compartments and collecting the barcode tagged nucleic acid fragments; sequencing the barcode and barcode tagged nucleic acid to characterize the accessible chromatin region on a single cell basis.

In one aspect, methods of barcoding the whole genome of a single cell are provided. The methods include providing a plurality of cells or nuclei and fixing the cells or nuclei to dissociate DNA from the proteins inside the cells or nuclei; providing a plurality of transpososomes, wherein each transpososome comprises at least one transposon and one transposase; incubating the fixed cells or nuclei and the transpososomes together to form strand transfer complexes (STCs) on DNA inside the fixed cells or nuclei; providing a plurality of unique barcode templates; compartmentalizing the treated nuclei and barcode templates to generate two or more compartments comprising both a cell or a nucleus and one or more than one barcode template with different barcode sequences; amplifying the barcode template in the compartment, breaking cellular and/or nuclear membrane, fragmenting the DNA by breaking the STCs to form tagmented nucleic acid fragments; attaching barcode sequences to tagmented nucleic acid fragments so that a plurality of fragments sharing the same one or more barcode sequences are presented in the compartment; removing the compartments; and collecting the barcode tagged nucleic acid fragments. In some embodiments, the strand transfer reaction occurs after a cell or nucleus is compartmentalized with the barcode template(s). In embodiments, the cells are prokaryotic or eukaryotic cells.

In one aspect, methods of barcoding a whole genome of a single cell are provided in which the methods include providing a plurality of cells or nuclei and fixing the cells or nuclei to dissociate DNA from the proteins inside the cells or nuclei; providing a plurality of transpososomes, wherein each transpososome comprises at least one transposon and one transposase; incubating fixed cells or nuclei and the transpososomes to form strand transfer complexes (STCs) on DNA inside the fixed cells or nuclei; providing a plurality of unique barcode templates; compartmentalizing the treated nuclei and barcode templates to generate two or more compartments which comprise both a cell or nucleus and one or more than one barcode template with different barcode sequences; attaching a barcode sequence to the genomic DNA in the cells or nucleus in the compartment by i) breaking the nuclear membrane, and fragmenting genomic DNA by breaking the STCs to form tagmented nucleic acid fragments; ii) amplifying the tagmented nucleic acid fragments and amplifying the barcode template; iii) linking a barcode template to a tagmented nucleic acid fragment, wherein a plurality of fragments sharing the same one or more barcode sequences are presented in the compartment; removing the compartments and collecting the barcode tagged nucleic acid fragments. In some embodiments, the strand transfer reaction occurs after a cell or nucleus is compartmentalized with the barcode template(s). In embodiments, the cells are prokaryotic or eukaryotic cells.

In one aspect, methods for single cell targeted sequencing are provided in which the methods include providing a plurality of cells and/or nuclei, providing a plurality of unique barcode templates and providing a plurality of target specific primers, wherein at least some of the target specific primers are also capable of attaching to the barcode templates directly or indirectly; compartmentalizing the cells and/or nuclei, the barcode templates and the target specific primers to generate two or more compartments comprising a cell and/or nucleus, one or more than one barcode template with different barcode sequences, and target specific primers; amplifying the barcode template in the compartment, attaching the barcode sequence to target specific primers, breaking the cell/nuclear membrane, priming target genomic regions with target specific primers to generate barcodes attached target fragments so that a plurality of barcodes attached target fragments sharing the same one or more barcode sequences are presented in the compartment; removing the compartments and collecting the barcode attached target fragments; and sequencing the barcode and barcoded tagged nucleic acid to characterize the targeted regions on a per cell basis. In embodiments, DNA, RNA, or both DNA and RNA are the target. When RNA is the target, reverse transcriptase is included in addition to a DNA polymerase.

In one aspect, methods for single cell targeted sequencing are provided in which the methods include providing a plurality of cells and/or nuclei; providing a plurality of unique barcode templates; and providing a plurality of target specific primers, wherein the target specific primers are capable of attaching to barcode templates directly or indirectly; compartmentalizing the cells and/or nuclei, wherein the barcode templates and the target specific primers generate two or more compartments comprising a cell and/or nucleus, one or more than one barcode templates with different barcode sequences and target specific primers; attaching a barcode sequence to a targeted nucleic acid fragment in the compartment by i) breaking cell and/or nuclear membrane to release nucleic acids; ii) amplifying the nucleic acid targets and amplifying the barcode template; iii) linking a barcode template to an amplified nucleic acid target, wherein a plurality of nucleic acid targets sharing the same one or more barcode sequences are presented in the compartment; removing the compartments and collecting the barcode-attached target fragments; and sequencing the barcode and barcoded tagged nucleic acid to characterize the targeted regions on a per cell basis. DNA, RNA, or both DNA and RNA are the target. When RNA is the target, reverse transcriptase is included in addition to a DNA polymerase.

In another aspect, methods for single cell RNA sequencing are provided in which the methods include providing a plurality of cells or nuclei, providing a plurality of unique barcode templates, providing a reverse transcriptase and providing a plurality of primers, which are capable of priming for cDNA synthesis, or for barcode template amplification, or for priming with cDNA, or for a combination thereof; compartmentalizing the cells, the barcode templates, the reverse transcriptase and the primers to generate two or more compartments comprise a cell, one or more than one barcode templates with different barcode sequences, reverse transcriptase and primers; lysing the cells, and generating cDNAs in the compartment, amplifying the barcode template, attaching the barcode sequence to cDNA fragments or fragments generated from cDNA so that a plurality of barcode attached fragments sharing the same one or more barcode sequences are presented in the compartment; removing the compartments and collecting the barcode attached fragments; and sequencing the barcode and barcoded tagged nucleic acid to characterize cDNA profile on a single cell basis. In an embodiment of the methods, unique molecular identifier (UMI) sequences can be incorporated in the primers for cDNA synthesis.

In another aspect, methods for single cell RNA sequencing are provided in which the methods include performing reverse transcription of RNA in situ; tagmenting cDNA in situ; compartmentalizing treated cells and barcode templates, wherein each compartment comprises one treated cell and one or more than one barcode templates; amplifying barcode templates and tagmented cDNA, and coupling amplified barcode templates to tagmented cDNA in the compartment; removing the compartments and collecting the barcode attached fragments; sequencing the barcode and barcoded tagged nucleic acid to characterize RNA profile on a single cell basis. In some embodiments, nuclei instead of cells are used as the input material.

In another aspect, methods for single cell RNA sequencing are provided in which the methods include providing a plurality of cells; fixing and/or permeabilizing the cells; providing a reverse transcriptase and providing a plurality of primers, wherein the primers are capable as primers for cDNA synthesis; generating first strand and second strand cDNA in situ; providing a plurality of transpososomes, each transpososome comprises at least one transposon and one transposase, tagmentating double-stranded cDNA in situ; providing a plurality of unique barcode templates: compartmentalizing the treated cells, the barcode templates, and the primers to generate two or more compartments comprising a cell, one or more than one barcode templates with different barcode sequences, and primers; in the compartment, amplifying the barcode template and cDNA fragments, attaching a barcode sequence to a cDNA fragment or fragment generated from cDNA so that a plurality of barcode attached fragments sharing the same one or more barcode sequences are presented in the compartment; removing the compartments and collecting the barcode attached fragments; and sequencing the barcode and barcoded tagged nucleic acid to characterize cDNA profile on a single cell basis. In some embodiments, nuclei instead of cells are used as the input material. In an embodiment, unique molecular identifier (UMI) sequences can be incorporated in the primers for cDNA synthesis.

In one aspect, methods for single cell RNA sequencing are provided in which the methods include providing a plurality of cells, fixing and/or permeabilizing the cells; providing a reverse transcriptase and providing a plurality of primers, wherein the primers are capable for use as primers for cDNA synthesis; generating first strand cDNA in situ; providing a plurality of transpososomes, each transpososome comprises at least one transposon and one transposase, tagmenting RNA/cDNA hybrid in situ; compartmentalizing the cells, the barcode templates, and the primers to generate two or more compartments comprising a cell or nucleus, one or more than one barcode templates with different barcode sequences, and primers; in the compartment, amplifying the barcode template and tagmented cDNA fragments, attaching said barcode sequence to cDNA fragments or fragments generated from cDNA so that a plurality of barcode attached fragments sharing the same one or more barcode sequences presented in the compartment; removing the compartments and collecting the barcode attached fragments; and sequencing the barcode and barcoded tagged nucleic acid to characterize cDNA profile on a single cell basis. In some embodiments, nuclei instead of cells are used as the input material. In an embodiment, unique molecular identifier (UMI) sequences can be incorporated in the primers for cDNA synthesis.

In one aspect, methods of analyzing both RNA and DNA in a single cell simultaneously are provided in which the methods include performing reverse transcription in situ for a plurality of cells, before or after cell fixation; performing strand transfer reaction in situ for the fixed cells; encapsulating these cells individually with one or more than one barcode template in a compartment; amplifying the barcode templates, cDNA and DNA fragments in the compartment; coupling amplified barcode templates to cDNA and DNA fragments in the compartment; removing the compartments and collecting the barcode attached fragments; sequencing the barcode and barcoded tagged nucleic acid to characterize both RNA and DNA profile on a single cell basis. In some embodiments, nuclei instead of cells are used as the input material.

In one aspect, methods of analyzing gene expression and gene regulation in a single cell simultaneously or RNA-seq and ATAC-seq in a single cell simultaneously are provided in which the methods include performing reverse transcription in situ on a plurality of cells; performing strand transfer reaction in situ for these cells; encapsulating these cells individually with one or more than one barcode template in a compartment; amplifying the barcode templates, cDNA and accessible chromatin DNA fragments in the compartment; coupling amplified barcode templates to cDNA and chromatin DNA fragments in the compartment; removing the compartments and collecting the barcode attached fragments; sequencing the barcode and barcoded tagged nucleic acid to characterize both RNA and accessible chromatin DNA profile on a single cell basis. In some embodiments, in situ strand transfer reaction is performed before the reverse transcription reaction. In some embodiments of the method, the cells are fixed before encapsulation.

In another aspect, methods of identifying the compartment origin of any barcodes when more than one barcode is present in a compartment when partitioning barcode templates and barcoding targets are provided. The methods include providing compartment content specific information, identifying both barcode information of a target and compartment content information of the barcode, and grouping the barcodes with the same compartment content information to collect all the targets associated with these barcodes.

In an embodiment, the compartment content information is shared breakpoint coordinates of tagmented fragments from more than one nucleic acid fragment, or shared UMI sequence from more than one target, or a combination thereof.

Compositions and articles described in the disclosure and embodiments herein were isolated or otherwise manufactured in connection with the examples provided herein. Other features and advantages of of the described disclosure and embodiments will be apparent from the detailed description, and from the claims.

### BRIEF DESCRIPTION OF THE DRAWINGS

FIG. 1 provides a schematic illustration of a nucleic acid barcoding method using transpososomes and barcode templates with a compartmentation reaction in accordance with the described methods. BC refers to a barcode on a barcode template.

FIGS. 2A-2D provide schematics illustrating methods to attach clonally amplified barcode templates to tagmented nucleic acid fragments in a compartment in accordance with the described methods. FIG. 2A, amplified barcode templates are used as primers to further amplify a target (200) in order to attach the barcode to the target in the compartment (201). FIG. 2B, a linker oligo (203) is used to couple amplified barcodes to a target (200) indirectly so that after amplification, a barcode sequence is attached to the target (202). FIG. 2C, dual amplification of a barcode template and a target (200) occurring in a compartment separately (204, 205), followed by coupling of an amplified barcode sequence to an amplified target (206, 207). FIG. 2D, dual amplification of two barcode templates and a target (200) occurring in a compartment separately (210, 213), followed by coupling of an amplified barcode sequence to an amplified target (214,215). BC refers to a barcode on a barcode template. BC1 and BC2 are different barcode sequences.

FIG. 3 provides a schematic illustrating a method for single cell ATAC-seq library preparation. The method involves using transpososomes to tag targets within nuclei and to couple targets to a plurality of barcode templates with a compartmentation reaction.

FIG. 4 provides a schematic illustrating a single cell whole genome barcoding method. The method involves using transpososomes to tag targets within fixed cell nuclei and to couple the targets to barcode templates with a compartmentation reaction.

FIG. 5 provides a schematic illustrating a method to enrich targeted regions using barcoded nucleic acid fragments and a target specific primer set.

FIG. 6 provides a schematic illustrating that a barcoded single cell, using the methods of the present disclosure, can provide significant improvements to the detection power of somatic mutations. The methods involve combining individual cell identification and sequencing error correction with unique molecule identification (UMI). As indicated, a low frequency mutant genotype can be identified from a mutant cell with minimal background signal from abundant normal cells after sorting with cell IDs and minimizing noise from sequencing error following correction with UMI.

FIG. 7 provides a schematic illustrating a single cell RNA-seq method. The method involves both in situ reactions and compartmentalized barcode amplification and coupling reactions.

FIG. 8 provides a flow chart illustrating a method to generate a single cell sequencing library for both 5′ end and 3′ end RNA-seq in the same cell.

FIG. 9 provides a schematic illustrating a single cell nucleic acid barcoding reaction for targeted sequencing in a compartment.

FIG. 10 provides a flow chart showing a sequencing library preparation workflow for same cell ATAC-seq and 3′ RNA-seq analysis.

FIG. 11 provides graphs showing 3′ single cell RNA-seq analyses using mock mixtures of human and mouse cells (1:1 ratio). These graphs support the single cell nature, low collision rates, and scalability of the methods of the present disclosure.

FIG. 12 provides graphs showing the uniform manifold approximation and projection (UMAP) visualization of 3′ single cell RNA-seq data. The graphs provide support that the methods of the present disclosure can resolve cell diversity within a complex mixture like human peripheral blood monocular cells (PBMC). The graphs also highlight the enhanced sensitivity of the methods of the present disclosure when identifying rare cell populations.

FIG. 13 provides graphs illustrating the profiling of full-length transcriptomic information in human Jurkat cells using the methods of the present disclosure.

FIG. 14 provides a schematic and graphs demonstrating genome-based identification and quantification of bacterial species within a mock mixture of five bacterial cells (1:1:1:1:1 ratios) using the methods of the present disclosure. In the graph in the rightmost panel, the bars representing bacteria are listed in the following order, from top to bottom, Klebsiella aerogene, Escherichia coli, Citrobacter freundii, Staphylococcus epidermis, Bacillus subtilis.

Transposases in the figures are showed as a tetramer or dimer which is for illustration only. Different transposases can be used in the reaction.

### DETAILED DESCRIPTION

Described and featured herein are improved methods for single cell nucleic acid detection and sequencing. This disclosure is based, at least in part, on the discovery that amplification of nucleic acid targets during tagmentation results in a number of advantageous benefits when compared to conventional sequencing techniques, including enhancing the detection of rare genetic variants and allowing for full-length sequencing of longer nucleic acid targets, while only using short read sequencing techniques.

Most commercially available sequencing technologies have limited sequencing read length. Second generation high throughput sequencing technologies can sequence only several hundred bases and rarely reach a thousand bases. However, nucleic acid sequences of a gene can span from several kilobases to tens and hundreds of kilobases, which means sequencing read length of tens of kilobases is necessary to successfully determine the haplotypes of all genes.

Currently, most sequencing methodologies involve bulk sequencing of DNA or RNA extracted from many cells at once, although individual cells are different. By using averaged molecular or phenotypic measurements of a cell population to represent an individual cell behavior, conclusions could be biased by the expression profiles of a majority group of cells or over-expressed outliers. In addition, such measurements lack the sensitivity to identify all unique patterns from an individual cell which could reflect distinctive functional behaviors for a cell at a given location and time. In addition, early tumor detection using current methodologies has been significantly restrained by a limited ability to detect a very low frequency of somatic mutation due to the presence of high background, wild type signal from normal cells or tissue. However, with the improved ability to identify every single cell as provided by the methods described herein, it mutant tumor cells can advantageously be separated from wild type or normal cells by genotyping at single cell level. Such methods will results in the removal of the wild type background signal generated from normal cells almost completely and make somatic mutation detection as easy as germline mutation detection.

Both Tn5 transpososomes and MuA transpososomes have been previously described to simultaneously fragment DNA and introduce adaptors at high frequency in vitro, creating sequencing libraries for next-generation DNA sequencing (Adey et al 2010, Caruccio et al 2011, and Kavanagh et al 2013). These specific protocols remove any phasing or contiguity information as a result of the fragmentation of the DNA. In these protocols after DNA reaction with transpososomes, a column purification, a heat treatment step, a protease treatment or an incubation with SDS solution or EDTA solution was necessary to release the transposase from the strand transfer complexes (STC) so that DNA is tagmented into fragments. It has been known that MuA transpososome can form a very stable STC when attacking DNA targets (Surette et al 1987, Mizuuchi et al 1992, Savilahti et al 1995, Burton and Baker 2003, Au et al 2004). Similar stability has also been observed for the Tn5 transpososome during a transposition reaction (Amini et al 2014).

In some embodiments, the present disclosure incorporates the stability of STCs, such as Tn5 transpososomes and MuA transpososomes, and clonal barcode generation by compartmentation amplification, to provide methods to uniquely barcode sub-fragments of nucleic acid targets and/or barcode nucleic acid targets in a single cell.

### Definitions

Unless defined otherwise, all technical and scientific terms used herein have the meaning commonly understood by a person skilled in the art to which this disclosure belongs. The following references provide one of skill with a general definition of many of the terms used in the disclosure and the embodiments therein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “adaptor” as used herein refers to a nucleic acid sequence that is added, for example, by ligation, to a nucleic acid. An adaptor can comprise a primer binding sequence, a barcode, a linker sequence, a sequence complementary to a linker sequence, a capture sequence, a sequence complementary to a capture sequence, a restriction site, an affinity moiety, unique molecular identifier, and a combination thereof.

The term “amplification” as used here refers to a process to generate multiple copies of an original template. The method for amplification may include processes such as PCR, RPA, MALBAC, and isothermal amplification methods for both linear amplification and exponential amplification.

The term “barcode template”, as used herein, refers to a barcode sequence, flanked by at least one handle sequence at one end, or two handle sequences at both ends. The length of a barcode sequence may range from 4 bases to 100 bases. The handle sequences can be used as binding sites for hybridization or annealing, as priming sites during amplification, or as binding sites for sequencing primers or transposase enzymes. Barcode sequences can be selected from a pool of known nucleotide sequences or can be randomly chosen from randomly synthesized nucleotide sequences. A barcode template can be a DNA, an RNA or a DNA/RNA hybrid.

By “biological sample” is meant any appropriate biological sample including blood and other liquid samples of biological origin including, but not limited to, peripheral blood, serum, plasma, cerebrospinal fluid (CFS), urine, stool, saliva, sputum, tears, lavage fluid, synovial fluid. The sample may include cells, tissue, organs or preparations thereof, obtained by procedures known and used in the art. The biological sample, cells, and/or nuclei of the present disclosure may be obtained, without limitation, from a mammal, non-human mammal, human, or non-mammal.

By “a cellular unit” is meant a single cell. A single cell under this definition includes both physical and virtual cells. For instance, a cellular unit may be a single cell, a single cell in a compartment, or the data representation of a single cell.

By “a compartment” is meant

By “de novo sequencing” is meant sequencing a novel genome where there is no reference sequence available for alignment. For example, sequence reads are assembled as contigs, and the coverage quality of de novo sequence data depends on the size and continuity of the contigs (ie, the number of gaps in the data).

By “haplotype phasing” or “haplotype estimation” is meant the determination of haplotypes, such as determining maternal and paternal haplotypes, from genotype data, such as from genomic DNA.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). “Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 3020 C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Primer set” refers to a set of oligonucleotides that may be used, for example, for polymerase chain reaction (PCR). In embodiments, a primer set can consist of at least 2, 4, 6, 8, 10, 12, 14, 16, 18, 20, 30, 40, 50, 60, 80, 100, 200, 250, 300, 400, 500, 600, or more primers.

The term “transposase” as used herein refers to a protein that is a component of a functional nucleic acid protein complex capable of transposition and which mediates transposition, including but not limited to Tn, Mu, Ty, and Tc transposases. The term “transposase” also refers to integrases from retrotransposons or of retroviral origin. A transposase can also refer to wild type protein, mutant protein and fusion protein with a tag, such as, GST tag, His-tag, etc. and a combination thereof.

The term “transposon”, as used herein, refers to a nucleic acid segment that is recognized by a transposase or an integrase and is an essential component of a functional nucleic acid-protein complex capable of transposition. Together with a transposase, a transposon forms a transpososome and performs a transposition reaction. “Transposon”, as used herein, refers to both wild type and mutant transposons.

A “transposable DNA” as used herein refers to a nucleic acid segment that contains at least one transposon unit. A transposable DNA may also comprise an affinity moiety, un-natural nucleotides and other modifications. The sequences besides the transposon sequence in the transposable DNA may also include adaptor sequences.

The term “transpososome” as used herein refers to a stable nucleic acid and protein complex formed by a transposase non-covalently bound to a transposon. A transpososome may comprise multimeric units of the same or different monomeric units.

A “transposon joining strand” as used herein means a strand of a double stranded transposon DNA that is joined by a transposase to a target nucleic acid at an insertion site.

A “transposon complementary strand” as used herein means the complementary strand of the transposon joining strand in the double stranded transposon DNA.

A “strand transfer complex (STC)” as used herein refers to a nucleic acid-protein complex including a transpososome and its target nucleic acid into which transposons insert, wherein the 3′ ends of the transposon joining strand are covalently connected to its target nucleic acid. An STC is a very stable form of nucleic acid and protein complex, which resists heat and high salt in vitro (Burton and Baker, 2003).

A “strand transfer reaction” as used herein refers to a reaction between a nucleic acid and a transpososome, in which strand transfer complexes (STCs) form.

A “tagmentation reaction” as used herein refers to a fragmentation reaction where transpososomes insert into a target nucleic acid through strand transfer reactions and form strand transfer complexes; the strand transfer complexes are then broken under certain conditions, such as, protease treatment, high temperature treatment, or a protein denaturing agent, e.g. SDS solution, guanidine hydrochloride, urea, etc., or a combination thereof, so that the target nucleic acid breaks into smaller fragments with a transposon attached to an end of the target nucleic acid (e.g., tagmented nucleic acid fragments). In general, tagmentation encompasses an initial step in the preparation of nucleic acid libraries in which unfragmented nucleic acid (e.g., DNA, cDNA, gDNA) is cleaved/broken and tagged for analysis.

A “reaction vessel” as used herein means a substance with a contiguous open space to hold liquid. In some embodiments, the reaction vessel is selected from the group consisting a tube, a well, a plate, a well in a multi-well plate, a slide, a spot on a slide, a droplet, a tubing, a channel, a bottle, a chamber and a flow-cell.

Preparation of a library for sequencing may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Encapsulating Nucleic Acid With Strand Transfer Complexes and Barcode Templates in Water-In-Oil Emulsion Droplets

The present disclosure provides methods to encapsulate nucleic acid targets in the form of strand transfer complexes (STCs) and barcode templates in water-in-oil emulsion droplets, to generate barcode tagged nucleic acid fragments.

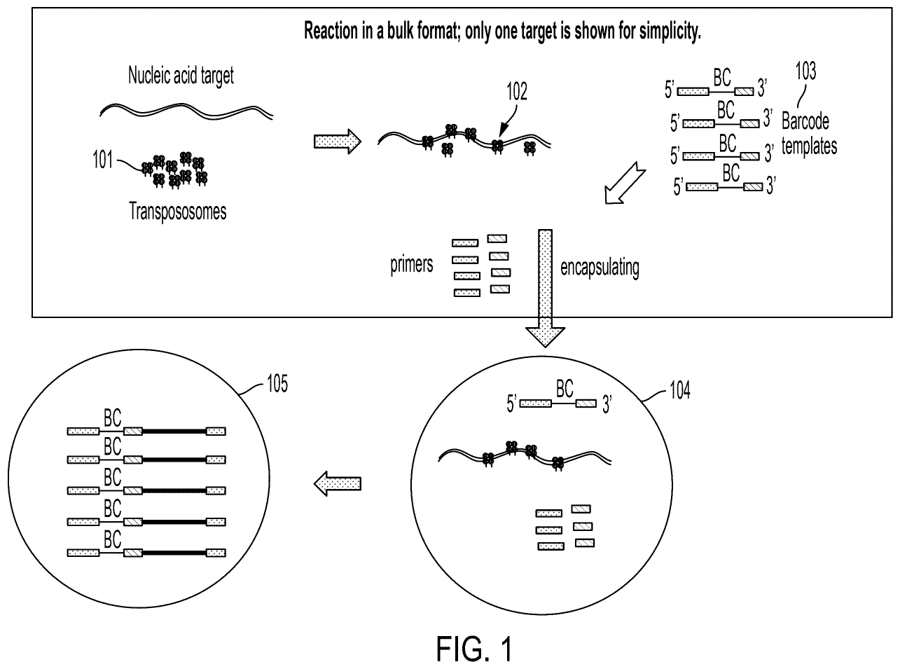

Nucleic acid targets are reacted with transpososomes (101) to stable strand transfer complexes (102) while keeping the contiguity of nucleic acid targets (FIG. 1). The nucleic acid targets may be double-stranded. In some embodiments, the nucleic acid targets are double stranded DNA. In some embodiments, the nucleic acid targets are DNA/RNA hybrids. Strand transfer reactions may involve a plurality of nucleic acid targets in one reaction vessel. In some embodiments, one type of transpososome (e.g., Tn5 or MuA) is used; in other embodiments, more than one type of transpososome is used simultaneously or sequentially (e.g., Tn5 and MuA). In an embodiment, the nucleic acid targets with STCs (102) are mixed with a plurality of barcode templates (103) in the solution. In some embodiments, each barcode template has a unique barcode sequence, which is different from the barcode sequence in another barcode template. In some embodiments, there are multiple populations of barcode templates, each having a unique barcode sequence that is different from the barcode sequences of others in the population, where each population includes at least one barcode template. In some embodiments, the barcode templates are oligonucleotides, existing freely in solution. In some embodiments, the barcode templates are arranged in a nanoball format. In some embodiments, the barcode templates are encapsulated in droplets. In some embodiments, the barcode templates are immobilized on a carrier, which can be a solid bead or particle (e.g., an nanoparticle), or a dissolvable bead or particle, or a combination thereof. In some embodiments, a carrier contains only a single barcode template. In some embodiments, a carrier comprises a plurality of barcode templates, where each template has a unique barcode sequence different from the barcode sequences of the other barcode templates. In some embodiments, a carrier contains only a single population of barcode templates, where the population of barcode templates has the same barcode sequence. In some embodiments, a carrier comprises multiple populations of barcode templates, each having a unique barcode sequence different from the barcode sequences of the others in the population, where each population includes at least one barcode template.

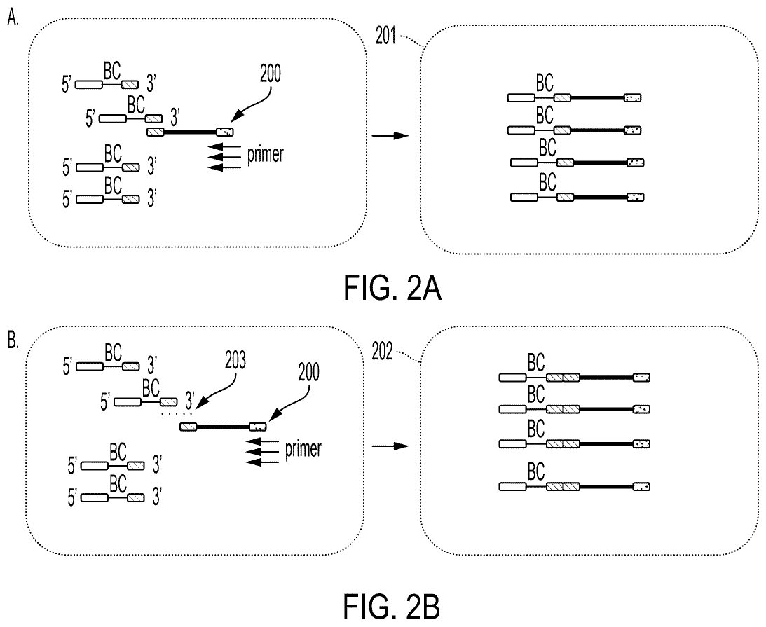

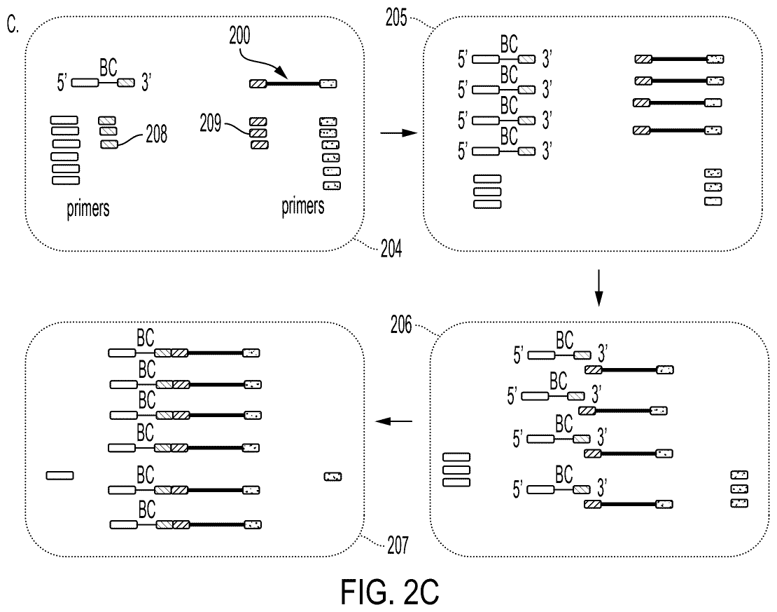

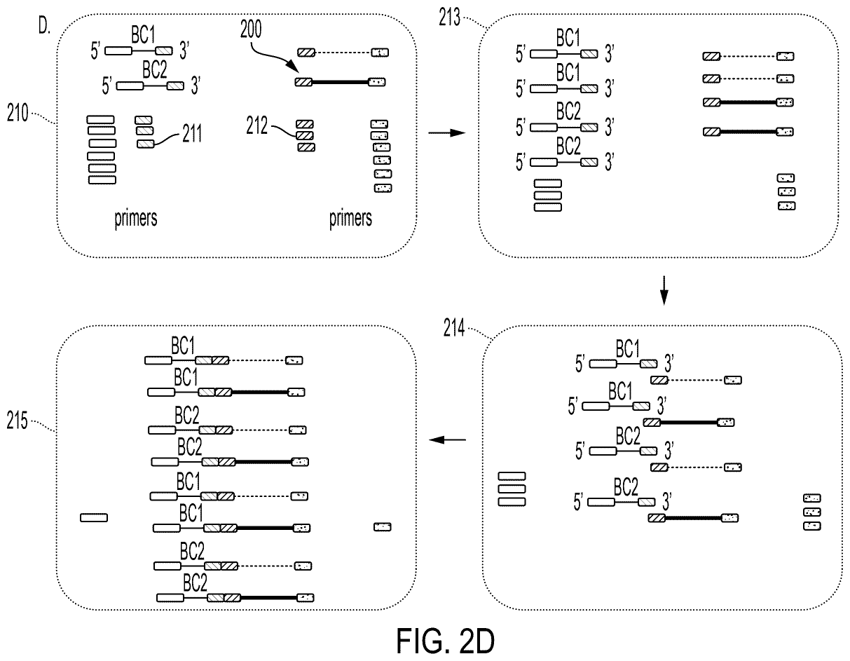

At least one of the transposable DNA in the transpososome is capable of hybridizing to one end of barcode template directly (FIG. 2A) or indirectly with a linker and/or a primer (FIG. 2B). Additional enzymes and substrates, such as, DNA polymerase, dNTP and primers are also provided in an aqueous solution in the same reaction vessel. In some embodiments, primers are used to amplify the barcode template. In some embodiments, primers can be used to amplify tagmented nucleic acid target fragments. Amplification includes exponential amplification and linear amplification. In some embodiments, different primers can be used to amplify the barcode template and tagmented nucleic acid target fragments in parallel (FIG. 2C); thereafter, the two groups of amplified products can merge/couple into one piece via shared homology between the two inner primers (FIG. 2C, 208 and 209) or via an additional linker which is capable of bridging a barcode template and a tagmented fragment together. Water-in-oil emulsion droplets (104) are generated under conditions in which one to a few nucleic acid targets with STCs are mixed with one barcode template in one droplet. Proper titration of nucleic acid targets with STCs and barcode templates can be used based on the Poisson distribution. In some embodiments, a plurality of barcode templates with different barcode sequences can be used in an emulsion droplet to significantly increase the presence of barcodes in the emulsion droplets, which advantageously increases the number of droplets with positive products, thus increasing the reaction yield of the barcoding reaction significantly.

In some embodiments, when both barcode templates and tagmented fragments are amplified before attaching a barcode sequence to a tagmented fragment, a plurality of barcode templates with different barcode sequences used in the same emulsion droplet will not affect the true representation of the nucleic acid targets, if different barcodes are randomly attached to the amplified copies of tagmented fragments (FIG. 2D). In this way, most emulsion droplets will contain barcode templates for barcoding nucleic acid targets when the barcode templates and nucleic acid targets are encapsulated in the same droplet. This makes it feasible to obtain nearly 100% of the droplets that contain any nucleic acid target which would be useful for barcoding. In embodiments, the emulsion droplets have a diameter of from 1 μm to 200 μm, or from 5 μm to 30 μm. When a plurality of barcodes are present in an emulsion droplet compartment, these barcodes can be traced to one original compartment by utilizing the breakpoint coordinates of the tagmented fragments. Specifically, the breakpoints created by transposase tagmentation are different among different nucleic acid targets. If DNA fragments attached with a barcode share the same breakpoint coordinates with fragments attached with one or more other barcodes, these fragments are likely to originate from the same original compartment. For a plurality of nucleic acid targets in an experiment, there is a possibility that two different nucleic acid fragments will produce the same breakpoint after transposase tagmentation. The chances for such collision are much lower when multiple breakpoints are used for discrimination. In some embodiments, unique molecular identifier (UMI) labeled transpososomes can be used during the strand transfer reaction or tagmentation reaction to increase the uniqueness of the fragment for identification. The UMI information can be used for compartment identification when different barcodes share many fragments with the same set of UMI population in addition to the same set of fragment breakpoints.

STCs are treated to release transposase from tagmented nucleic acid target fragments, for example, by heat treatment. After heat treatment, such as, for example, at 60° C. to 75° C. for about 5-10 minutes, the transposase will be released from the STCs and the nucleic acid target will break into smaller fragments. In some embodiments, while still in the emulsion droplet, a DNA polymerase fills in the gaps left during the transposition reaction. Emulsion amplification is performed to amplify the barcode templates in the droplet. Amplified barcode templates will hybridize to the tagmented fragments directly (FIG. 2A) or indirectly (FIG. 2B) and attach the barcode sequence to the fragments (105, 201, and 202) during the amplification reaction. In some embodiments, unique molecular identifiers (UMIs) are added to the barcode templates during the emulsion reaction. In some embodiments, UMIs are integrated as a linker (203) or a primer (209 and 212) in FIG. 2. After the emulsion amplification reaction, emulsion droplets are dispersed, for example, by high salt, detergent, alcohol, organic chemicals, or combination thereof. After the emulsion droplets are dispersed, the aqueous phase of the resulting solution is collected. In some embodiments, one or more biotinylated primers are used so that amplified barcoded fragments can be readily bound to streptavidin beads. In some embodiments, one or more biotinylated dNTPs are used in the emulsion amplification. In some embodiments, primers with sample-specific barcodes are used in the emulsion droplets during emulsion amplification so that emulsion amplification products from different sample reactions can be pooled together for final amplification or adaptor modification to make sequencing ready libraries.

In some embodiments, the nucleic acid target is whole genomic DNA. This barcoding method can be used for de novo sequencing, whole genome haplotype phasing and structural variant detection. In some embodiments, the nucleic acid targets are DNA fragments, cDNA, or a portion of captured DNA by hybridization capture, primer extension or PCR amplification. This barcoding method can phase the variants of these DNA molecules. In some embodiments, target specific primers can be used in the compartment to amplify specific nucleic acid targets with or without reaction with transpososomes.

### Encapsulating Transposase Tagged Cells or Nuclei and Barcode Templates in Water-In-Oil Emulsion Droplets

Described herein is a method to encapsulate cells or nuclei after strand transfer reaction and a barcode template in water-in-oil emulsion droplets, and further to generate barcode tagged nucleic acid fragments for single cell level analysis.

Assay for Transposase-Accessible Chromatin using sequencing (ATAC-seq) is gaining greater popularity as a state-of-the-art molecular biology tool to assess genome-wide chromatin accessibility (Buenrostro et al, 2013). ATAC-seq identifies accessible chromatin regions by tagging open chromatin with a hyperactive mutant Tn5 transposase that integrates sequencing adaptors into open regions of the genome. The tagged DNA fragments are purified, amplified by PCR and sequenced. Sequencing reads are then used to infer regions of increased accessibility, as well as to map regions of transcription-factor binding sites and nucleosome positions. While natural wild type transposases have a low level of activity, ATAC-seq employs a mutated hyperactive transposase (Reznikoff et al, 2008), which has been successfully adapted to efficiently identify open chromatin and identify regulatory elements across the genome. Furthermore, single cell ATAC-seq is used to separate single nuclei and perform ATAC-seq reactions individually (Buenrostro et al, 2015). Higher throughput single cell ATAC-seq uses combinatorial cellular indexing to measure chromatin accessibility in thousands of individual cells. Single-cell ATAC seq enables the identification of cell types and states for developmental lineage tracing. ATAC-seq will likely be a key component of comprehensive epigenomic workflows.

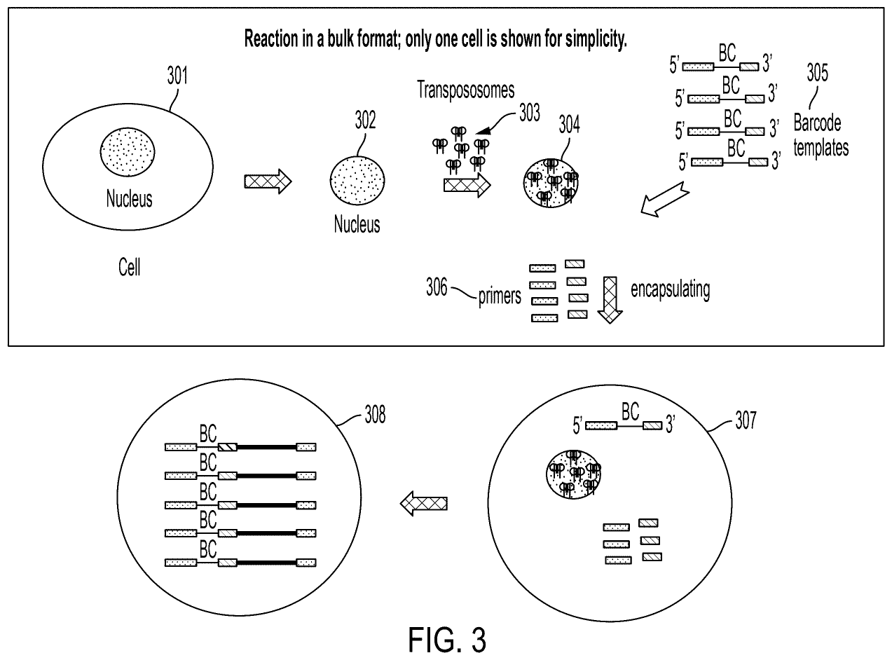

In some embodiments, the present disclosure includes methods using emulsion of water-in-oil droplets to encapsulate a transposase treated nucleus and a unique barcode template. The method also involves clonally amplifying the barcode template within the emulsion droplet and attaching the clonally amplified barcodes to tagmented accessible DNA fragments (FIG. 3). The tagmented DNA can also be amplified in the emulsion droplet. The barcoding methods of the present disclosure offer the advantages of high throughput and low-cost cellular indexing, for single cell ATAC-seq analysis.

In some embodiments, nuclei (302) are collected from cells or tissue samples (301) and incubated with transpososomes (303) to form STCs (304), which are then mixed with a plurality of barcode templates (305) in a bulk reaction (FIG. 3). In some embodiments, each barcode template has a unique barcode sequence, which is different from the barcode sequence in other barcode templates. In some embodiments, there are multiple populations of barcode templates, each having a unique barcode sequence different from the barcode sequence of the other barcode template populations, where each population includes at least one barcode template. In some embodiments, the barcode templates are oligonucleotides existing freely in solution. In some embodiments, the barcode templates are arranged in a nanoball format. In some embodiments, the barcode templates are encapsulated in droplets. In some embodiments, the barcode templates are immobilized on a carrier, which can be a solid bead or particle (e.g., a nanoparticle), or a dissolvable bead or particle, or a combination thereof. In some embodiments, a carrier contains only a single barcode template. In some embodiments, a carrier comprises a plurality of barcode templates, where each template has a unique barcode sequence different from the barcode sequences of each other barcode template. In some embodiments, a carrier contains only a single population of barcode templates, where the population of barcode templates has the same barcode sequence. In some embodiments, a carrier comprises multiple populations of barcode templates, each having a unique barcode sequence different from the barcode sequences of the other barcode template populations, where each population includes at least one barcode template.

In some embodiments, whole cells are treated with transpososomes to form STCs inside the nuclei without the isolation of nuclei. In some embodiments, the transpososome comprises a mutated hyperactive Tn5 transposase. In some embodiments, the transpososome comprises a MuA transposase. Other enzymes and substrates, such as DNA polymerase, dNTP and primers (306) may also be provided in an aqueous solution in the same bulk reaction. Water-in-oil emulsion droplets are generated under conditions such that one nucleus and one barcode template are present in most droplets by limiting titration or partitions based on Poisson distribution (307). In embodiments, the emulsion droplets have a diameter of from 10 μm to 200 μm, or from 20 μm to 60 μm.

STCs are treated to release transposase from tagmented nucleic acid target fragments, for example, by heat treatment. After heat treatment, such as, at 60° C. to 75° C. for about 5-10 minutes, the transposase will be released from the STCs and the nucleic acid target breaks into smaller fragments. In some embodiments, while still in the emulsion droplet, a DNA polymerase present in the droplet fills in the gaps left during the transposition reaction. The nuclear membrane breaks during the emulsion PCR denaturing step, and emulsion amplification is performed to amplify the barcode templates in the droplet. Amplified barcode templates are capable of hybridizing to the tagmented fragments directly or indirectly and attaching the barcode sequence to the fragments during the amplification reaction (308). In some embodiments, both barcoded templates and tagmented fragments are amplified in parallel first, and then are merged or coupled together to form barcoded tagmented fragments as illustrated in FIGS. 2C and 2D. After the emulsion amplification reaction, emulsion droplets are dispersed, for example, by high salt, detergent, alcohol, organic solution or combination thereof. After the emulsion droplets are dispersed, the aqueous phase of the resulting solution is collected. In some embodiments, one or more biotinylated primers or one or more biotinylated dNTPs are used so that amplified barcoded fragments can be readily bound to streptavidin beads. In an embodiment, a sequencing library prepared from these barcoded fragments is a single cell ATAC-seq library.

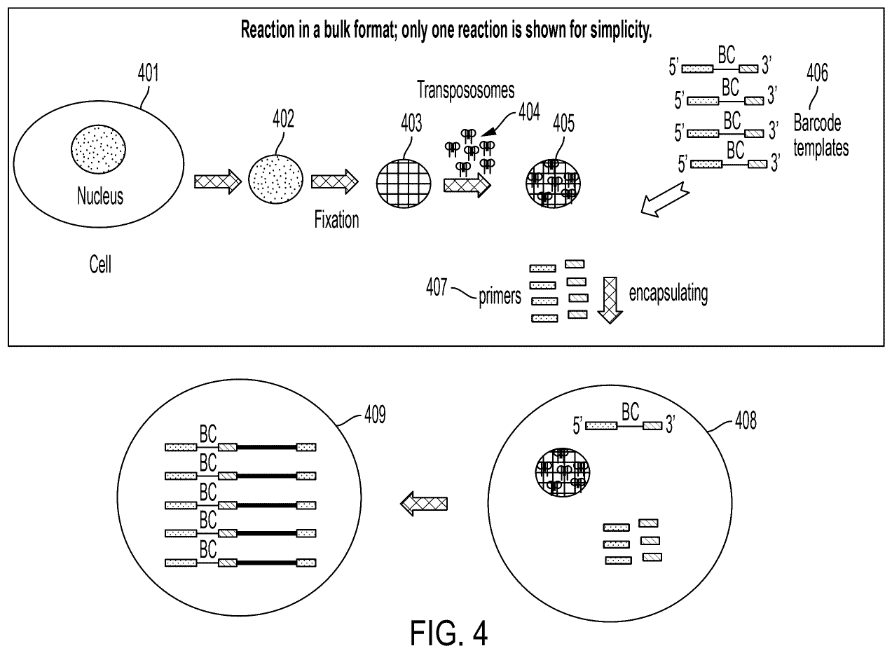

The present disclosure also provides a single cell whole genome sequencing method as described herein. The method employs emulsions to encapsulate an alcohol-fixed nucleus that is treated with transposase and a unique barcode template. The method also involves clonally amplifying the barcode template within the emulsion droplet and attaching the barcodes to tagmented genomic DNA fragments from the fixed nucleus (FIG. 4).

In some embodiments, nuclei (402) are collected from cells or a biological sample, such as a tissue sample (401) and fixed. Fixatives, such as an alcohol based fixative or a Hepes-glutamic acid buffer-mediated organic solvent protection effect (HOPE) fixative, or other similar fixatives may be used in these methods to stabilize/denature the proteins in the nuclei while keeping the nucleic acid contents of the nucleus intact (403). In some embodiments, fixation exposes all of the genomic DNA from the chromatin in the nucleus. In some embodiments, fixed cells are used directly without the isolation of nuclei. After washing away the fixation solution, nuclei are treated with transpososomes (404) to form STCs (405) with the genomic DNA, and then are mixed with a plurality of different barcode templates (406) in a bulk reaction. Other enzymes and substrates, such as, DNA polymerase, dNTP and primers (407) are also provided in an aqueous solution in the same bulk reaction. Water-in-oil emulsion droplets are generated under conditions such that one nucleus and one barcode template are present in a droplet by limiting titration or partitions based on Poisson distribution (408). In an embodiment, the emulsion droplets have a diameter from 10 μm to 200 μm, or from 20 μm to 60 μm.

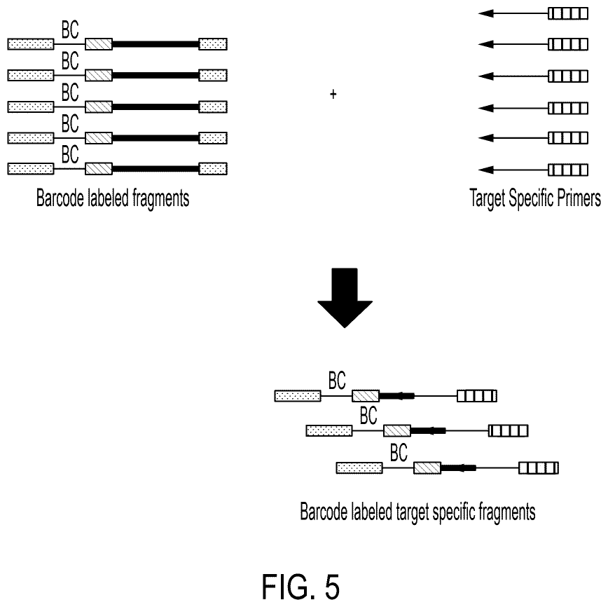

STCs are treated to release transposase from tagmented nucleic acid target fragments, for example, by heat treatment. After heat treatment, such as, at 60° C. to 75° C. for about 5-10 minutes, transposase will be released from the STCs and the nucleic acid target is broken into smaller fragments. In some embodiments, while still in the emulsion droplet, a DNA polymerase present in the droplet fills in the gaps left during the transposition reaction. The nuclear membrane of the nucleus is broken, and emulsion amplification is performed to amplify the barcode templates in the droplet. Amplified barcode templates are capable of hybridizing to the tagmented fragments directly or indirectly and attach the barcode sequence to the fragments during the amplification reaction (409). In some embodiments, both barcoded templates and tagmented fragments are amplified in parallel first, and then are merged together to form barcoded, tagmented fragments as in FIGS. 2C and 2D. After the emulsion amplification reaction, emulsion droplets are dispersed, for example, by high salt, detergent, alcohol, organic reagents or combination thereof. After the emulsion droplets are dispersed, the aqueous phase of the resulting solution is collected. In some embodiments, one or more biotinylated primers or one or more biotinylated dNTPs are used so that amplified barcoded fragments can be pulled out easily with streptavidin beads. In some embodiments, library prepared from these barcoded fragments can be used directly for single cell whole genome sequencing and single cell copy number variation (CNV) analysis. In some embodiments, a library prepared from these barcoded fragments can be used for further targeted capture of whole exomes or for targeted capture of smaller targeted regions for targeted sequencing (FIG. 5). In some embodiments, cells from a metagenomic sample are used in this barcoding reaction directly. In some embodiments, prokaryotic cell walls can be permeabilized enzymatically and/or chemically.

Advantageously, the single cell sequencing methods of the present disclosure eliminate the need for genomic DNA preparation, which is a known bottleneck for metagenomic sample preparation, while keeping high molecular weight DNA intact in the cells directly to improve assembly efficiency. The methods of the present disclosure preserve the composition of the organism in a metagenomic sample very well and improve the accuracy of the measurement of organism composition using cell level information based on barcodes, instead of only genomic DNA level information, which contains more bias due to accessibility, amplification, or sequencing.

In some embodiments, the cells are microbes. In some embodiments, the cells are microbiome cells or metagenomic cells. In some embodiments, microbial or metagenomic samples are pretreated with lysozyme or other cell wall lysis enzymes to facilitate the removal of the cell wall as part of the preparation. In some embodiments, the methods as described are used to analyze metagenomic or microbiome samples for sample species identification, composition analysis and microbial host and their plasmids or bacteriophage or virus association.

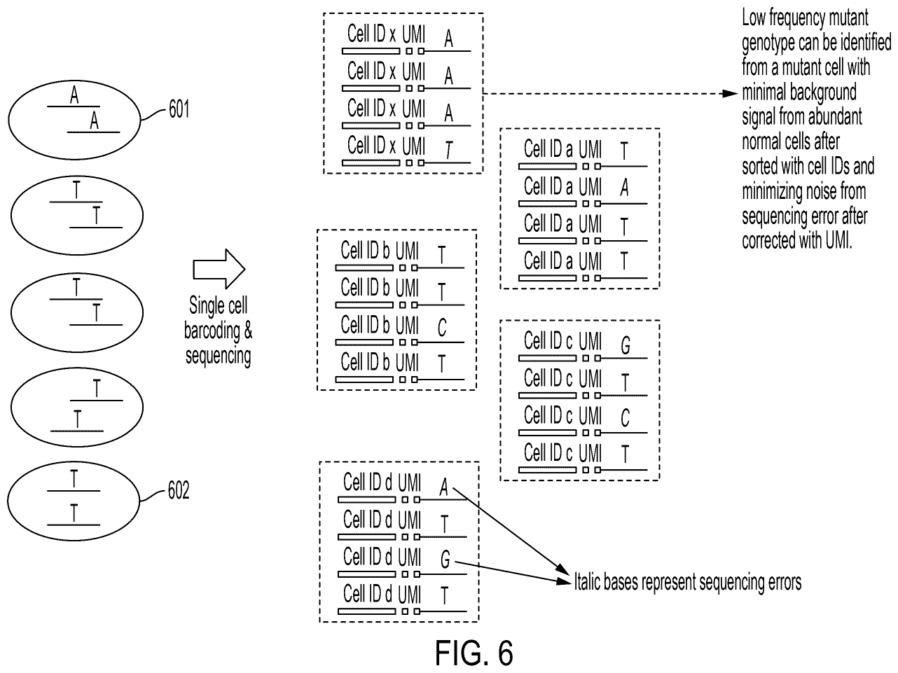

One advantage of the single cell targeted barcoding and/or sequencing methods disclosed herein is that they have much higher sensitivity for the detection of low frequency genetic variants, such as, detection of somatic mutations (FIG. 6), when compared to known barcoding and/or sequencing methods. Since the present methods allow for the unique barcoding of individual cells, it is possible to detect any mutations at a single cell level, which will effectively eliminate the background noise from surrounding cells. This provides very high sensitivity for detecting very low frequency somatic mutations, such as is required for early cancer detection. FIG. 6 illustrates the advantages of genotyping at a single cell level. Shown in FIG. 6 is a cell containing a mutant allele A (601), but in the presence of many wild type cells containing a normal allele T (602) in the same sample. Unique molecular identifiers (UMIs) are added in the barcoding reactions. With the incorporation of molecule specific UMIs during single cell barcoding and sequencing, sequencing reads can be grouped based on their cell ID first, and for each cell, it is possible to identify sequencing error based on UMI and make a correct variant call easily. This approach can be applied for circulating tumor cells, tissue biopsy samples or tissue sections.

In some embodiments, a plurality of barcode templates with different barcode sequences can be presented in an emulsion droplet to increase the capture rate. When a plurality of barcode templates are present in the emulsion droplet and shared by one nucleus or cell, these barcodes can be traced back to their original nucleus or cell by utilizing the breakpoint coordinates of the tagmented fragments. Specifically, the breakpoints created by transposase tagmentation are different among different nuclei or cell. If DNA fragments attached with a barcode share the same breakpoint coordinates with fragments attached with one or more other barcodes, these barcodes are likely to originate from the same original nucleus or cell. There is a possibility that two nuclei or cells will produce the same breakpoint in some fragments after transposase tagmentation. The chances for such collision are much lower when multiple breakpoints are used for discrimination. The more shared breakpoint coordinates among two barcodes, the higher is the confidence that these two barcodes are from the same compartment, i.e. the same cell or nucleus. In some embodiments, the randomness of the tagmentation breaking point is used as a UMI function to track duplication that has arisen from the amplification and to improve the counting accuracy of unique targets.

When a plurality of different barcode templates is present in a droplet which captures a cell or a nucleus, in combination with the subsequent amplification of the barcode templates and tagmented cellular contents, additional copies of the same cellular content are created, either DNA, RNA or other cellular targets, and these additional copies are coupled to different barcode templates randomly. When multiple copies of cellular content are shared and captured randomly among different barcode templates in one droplet, so that each barcode template (or population of templates) can capture sufficient cellular content to represent the cell or nucleus in the droplet, this may effectively amplify the signal from one cell, creating “amplified” copies of a single cell. Although there is only one cell or nucleus in the droplet, multiple barcode templates in the droplet create multiple cells or nuclei representing the same cell or nucleus after the amplification in the droplet. This amplification of single cells can improve the downstream clustering analysis for cell population characterization and increase the assay sensitivity for detection of rare cell populations with low number of input cells or nuclei in a single cell reaction. The methods of the present disclosure provide new single cell library methods capable of amplifying single cells for use in the field.

In some embodiments, the methods of the present disclosure may also be used for single cell RNA analysis. In some embodiments, a reverse transcriptase and cDNA primers as the first set of primers can be included in the emulsion reaction. In some embodiments, the cDNA primers include a poly T sequence at the 3′ end; in some embodiments, the cDNA primers have a GGG nucleotide sequence at the 3′ end; in some embodiments, the cDNA primers have target specific primers at the 3′ end. In some embodiments, cDNAs are synthesized using mRNA as templates; in some embodiments, cDNAs are synthesized using other RNA species as templates. During the early phases of the emulsion reaction, cDNA or partial cDNA is generated from mRNA in the single cell or nucleus by reverse transcriptase. Barcoding then proceeds as described in any of the previously described methods, except using the cDNA as the input DNA. With different primers used for reverse transcription or cDNA priming, this method can be modified for single cell transcriptome analysis, single cell 3′ RNA-Seq analysis, single cell 5′ RNA-Seq analysis, single cell target-seq application, and immune repertoire analysis. Methods of the present disclosure may combine in situ reactions for bulk cells and encapsulation of individually treated cells with one or more barcode templates for compartmentalized amplification and barcode tagging reactions, thus allowing for high throughput single cell RNA analysis.

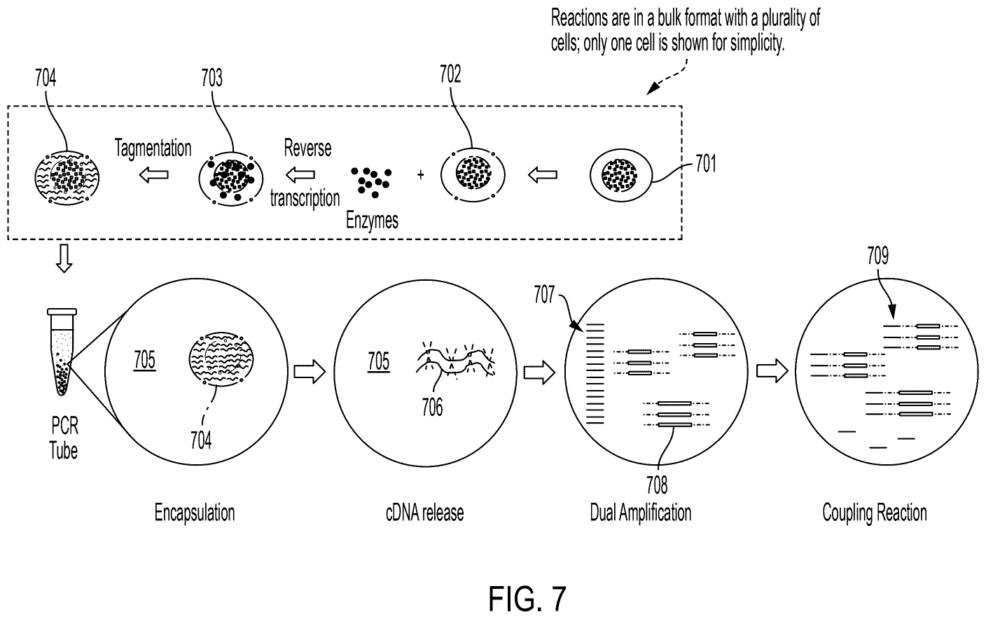

FIG. 7 illustrates one embodiment of a method of single cell RNA barcoding, according to the present disclosure. Cells (701) are first permeabilized (702). In some embodiments, RNAs in the permeabilized cells (702) are transcribed to cDNAs by reverse transcriptase in situ (703). A second strand of DNA is synthesized to form a double-stranded DNA as input for tagmentation in situ. In some embodiments, RNAs in the cells are transcribed to first strand cDNAs by reverse transcriptase in situ. RNA/cDNA hybrid double strand may also be used as input for tagmentation in situ (704). In some embodiments, the cDNA primers have a poly T sequence at the 3′ end; in some embodiments, the cDNA primers have a GGG sequence at the 3′ end; in some embodiments, the cDNA primers have target specific primers at the 3′ end; in some embodiments, cDNAs are synthesized using mRNA as templates; in some embodiments, cDNAs are synthesized using other RNA species as templates. The treated cells containing in situ tagmented cDNA (704) are encapsulated with one or more barcode templates (705) for a clonal amplification reaction. During the clonal reaction, tagmented cDNA fragments (706) are released from the cells, both barcode template(s) and tagmented cDNA are amplified (dual amplification) and amplified barcode templates (707) are coupled to the amplified cDNA fragments (708) and a plurality of barcode attached fragments sharing the same one or more barcode sequences presented in the compartment are generated (709). With different primers used for reverse transcription or cDNA priming, this method can be modified for single cell transcriptome analysis, single cell 3′ RNA-Seq analysis, single cell 5′ RNA-Seq analysis, single cell target-seq application, and immune repertoire analysis.

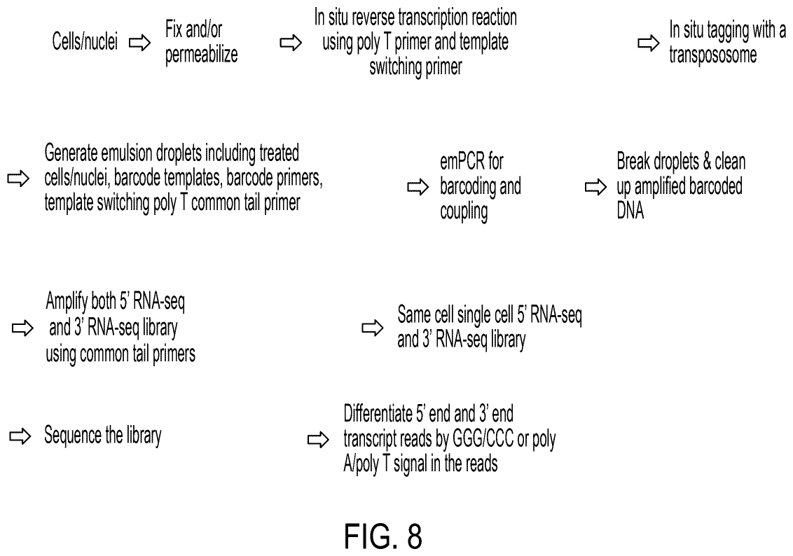

In some embodiments, both 3′ end RNA and 5′ end RNA targets can be captured in the same assay for the same cell as simultaneous 3′ RNA-seq and 5′ RNA-seq analysis (FIG. 8). In some embodiments, the full-length transcripts can be captured for full-length single cell transcriptome analysis using tagmented cDNA/RNA hybrids or double stranded cDNAs. Full-length transcripts and/or transcriptome analysis is very useful to study alternative splicing and mRNA isoforms. In some embodiments, optimized fixation and/or permeabilization condition are designed to drive the reaction to cytoplasmic mRNA mainly for full-length transcriptome analysis to reduce the representation of precursors mRNA and genomic or chromatin DNA in the nucleus. The single cell full-length transcriptome method as described herein is particularly suitable for use with short-read sequencing platforms because the process can break long full-length transcripts into multiple short fragments and coupled to the same barcode templates in a cellular unit. The short transcript fragments are easy to amplify, especially as compared to longer sequences, and the final library length can be relatively short. The resulting library is well adapted for use with short-read sequencing platforms. In some embodiments, this method can be used for long-read sequencing platform when keeping the tagmented transcripts long.

In some embodiments, a plurality of barcode templates with different barcode sequences can be presented in an emulsion droplet to increase the cell capture rate. When a plurality of barcode templates are present in an emulsion droplet and shared by one cell or nucleus in the compartment, these barcodes can be traced to one original cell/nucleus by the UMI on the reverse transcription primer or by analysis of the unique tagmentation breaking points on the transcripts. In some embodiments, it is preferable to keep these barcodes as (virtual) separate cells and not to merge these different barcodes back to their original cell origin. In this case, one cell or nucleus may be amplified into multiple cells or nuclei after the reaction. These amplified cells can improve the downstream clustering analysis for cell population characterization and increase the assay sensitivity when detecting rare cell populations with a low number of input cells or nuclei in a single cell reaction.

### Encapsulating Cells, Barcode Templates and Target-Specific-Primers in Water-In-Oil Emulsion Droplets

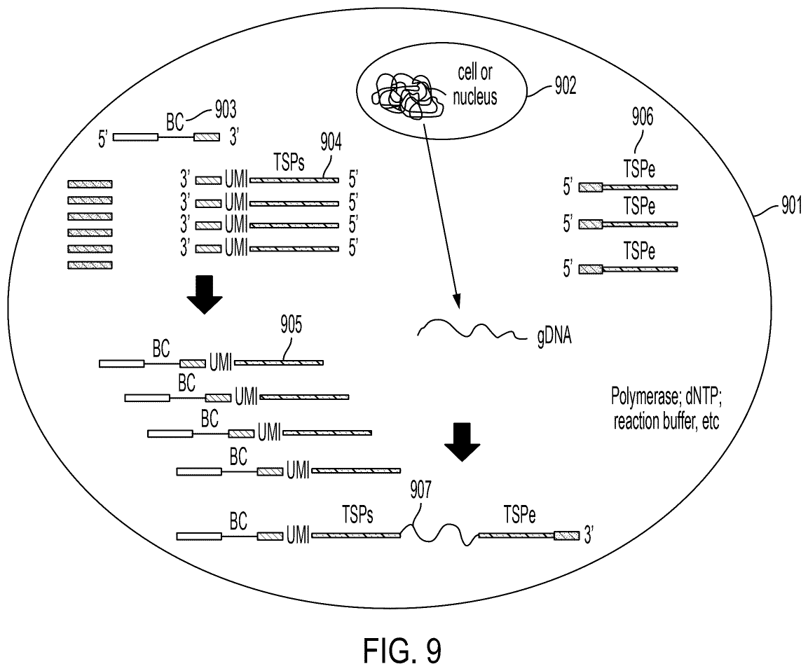

The present disclosure also provides a high throughput method for single cell targeted sequencing. FIG. 9 illustrates one embodiment of this high throughput method. Isolated cells or nuclei (902) may be encapsulated with unique barcode templates (903) and a first set of target specific primers (904) within emulsion droplets (FIG. 9, 901). Additional enzymes and substrates, such as, DNA polymerase, dNTP and common primers may also be provided in the aqueous solution. Water-in-oil emulsion droplets (901) are generated in such conditions that one cell or one nucleus and one barcode template are present in a droplet by limiting titration or partitions based on Poisson distribution. In an embodiment, the emulsion droplets have a diameter from 10 μm to 200 μm, or from 20 μm to 100 μm. The cell membrane and/or nuclear membrane is broken to release genomic DNA into the emulsion droplets. An emulsion amplification reaction is performed to amplify the barcode template and attach target specific primers to the barcode template in the droplet. Single stranded amplified barcode templates with target specific sequences at the 3′ end (905) are capable of hybridizing to genomic DNA targets and making copies of the targeted region during the emulsion amplification reaction. In some embodiments, a second set of target specific primers (906) is included in the aqueous solution during emulsion droplet generation. After the emulsion amplification reaction, barcode tagged amplicons of the targets (907) will be generated, which can be used for sequencing library preparation and sequencing analysis. In some embodiments, to reduce primer dimers generated during amplification, dUTP containing primers can be used and in combination with UDG/APE1/Exol treatment after emulsion amplification. Sequencing library adaptors can be added by ligation after cleaning up primer dimers.

### Method for Analyzing RNA and DNA in the Same Cell

Currently most single cell analysis methods are only capable of separately analyzing RNA or DNA for different single cells. In other words, currently known single cell analysis methods do not analyze both RNA and DNA from the same cell at the same time.

However, the methods of the present disclosure include monitoring RNA expression and determining DNA genotype for the same cell simultaneously. In some embodiments, cells after an in situ reverse transcription reaction to generate cDNA, are fixed to dissociate DNA from protein and/or stabilize the product. In some embodiments, cells are fixed first before performing an in situ reverse transcription reaction. Poly T primers can be used to capture 3′ mRNA. In some embodiments, a UMI sequence is associated with the poly T primers. A strand transfer reaction or tagmentation reaction can be performed in situ inside the treated cells or after the cells are encapsulated with barcode templates in a compartment. In some embodiments, a strand transfer reaction or tagmentation reaction is not necessary if the nucleic acid targets are all specific. During cell encapsulation in the compartment, cDNA specific primers and DNA target specific primers and/or transposon specific primers are included with primers for amplifying barcode templates at the same time. In some embodiments, cDNA amplification is for 3′ mRNA when using poly T primers. In some embodiments, DNA amplification is target specific or is whole genome specific. After amplification of barcode template(s) and cDNA and/or DNA fragments, barcode templates are linked to amplified cDNA and/or DNA fragments in the compartment. Barcode tagged cDNA and DNA are then released from the compartment and collected for further analysis on gene expression and genomic variation.

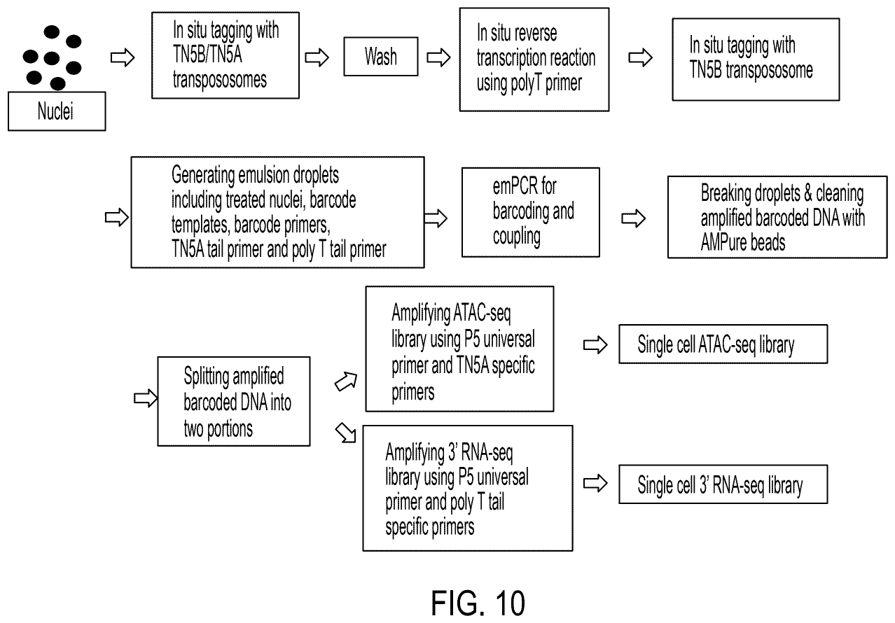

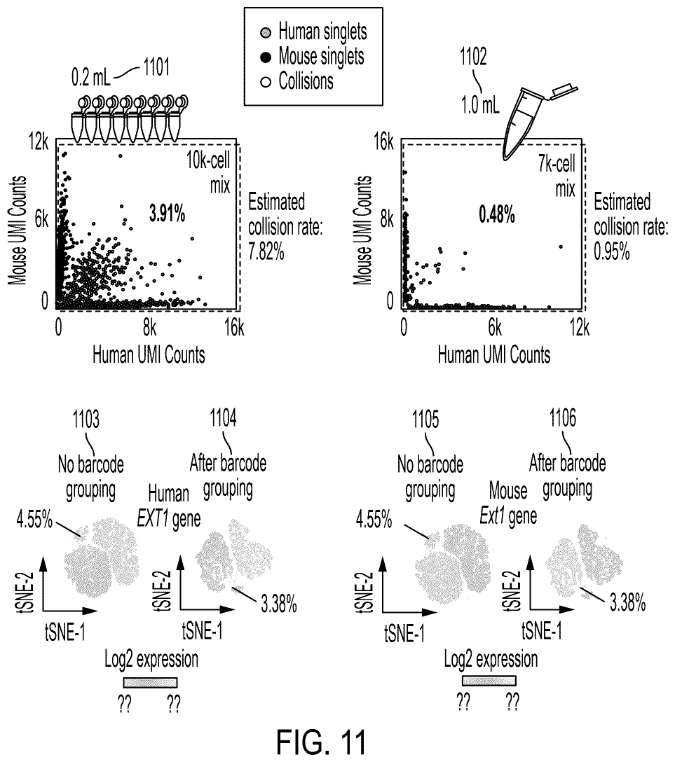

The present disclosure also provides a method for simultaneous ATAC-seq and RNA-seq of the same cell. Cells are permeabilized and reverse transcription using poly T labeled primers to generate cDNA are performed in situ. In some embodiments, the cDNAs are generated after first strand cDNA only. In some embodiments, the cDNAs are generated after second strand cDNA synthesis. The cells are incubated with transpososomes for strand transfer reaction at open chromatin sites inside the nuclei and with cDNA in the cells. In some embodiments, strand transfer reaction at open chromatin sites is performed before reverse transcription. The cells are then encapsulated in compartments, individually with one or more barcode templates in a compartment for barcode amplification and tagmented RNA and DNA amplification. In some embodiments, these cells are fixed to denature cellular proteins and exogenous reverse transcriptase and transposase before encapsulation. In some embodiments, nuclei are isolated from cells before the strand transfer reaction and/or reverse transcription reaction (FIG. 10).

Cellular Indexing of Transcriptomes and Epitopes by Sequencing (CITE-seq) is a multimodal single cell phenotyping method, which uses DNA-barcoded antibodies to convert detection of proteins into a quantitative, sequencable readout. Antibody-bound oligos act as synthetic transcripts that are captured during most large-scale oligo dT-based single cell RNA-seq library preparation protocols (Stoeckius et al, 2017). In some embodiments, when the cDNA primer is labeled with a polyT sequence, CITE-seq libraries are able to be generated efficiently.

In some embodiments, instead of a nucleic acid, a genome, a protein, a nucleus, a cell or a microbe, the encapsulated target is a protein complex, a protein and nucleic acid complex, a small molecule, a macromolecule, a chemical compound, a ligand, a particle, a microparticle, or a combination thereof. The encapsulated targets may be labeled with or attached to a nucleic acid as an identifiable label or marker.

In some embodiments for the methods of the present disclosure, the cells are eukaryotic cells; in other embodiments, the cells are prokaryotic cells.

Encapsulation in a water-in-oil emulsion is one method of compartmentation (sequestration) used in the methods of the present disclosure, but other sequestering methods are also feasible and may be used in the described methods. Certain types of liposomes, such as, giant unilamellar liposome vesicles (GUVs) with a size from 1-200 um in diameter, have shown very high thermostability and are able to perform PCR amplification inside of its enclosure (Kurihara et al 2011, Laouini et al 2012). Accordingly, in some embodiments, GUVs may be used as compartments in the present methods. In some embodiments, compartmentation is achieved by microwells. In some embodiments, compartmentation is achieved by open array. In some embodiments, compartmentation is achieved by microarray, microtiter plate or other physically separated compartmentation methods.

An embodiment is directed to a method of analyzing and/or counting nucleic acids from single cells, in which the method involves (a) providing a sample comprising a cell within a plurality of cells, wherein the cell comprises a plurality of sample nucleic acids; (b) generating a plurality of barcoded polynucleotides from the plurality of sample nucleic acids of said cell, wherein the barcoded polynucleotide comprises a barcode sequence configured to distinguish said sample nucleic acid from other sample nucleic acids in other cells; and a sample sequence from the sample nucleic acid in the cell, wherein said sample sequence comprising a distinguishable sequence from other sample sequences of other sample nucleic acids in said cell; (c) sequencing said barcoded polynucleotide to determine the sample sequence and the barcode sequence; (d) analyzing and/or counting sample nucleic acids in said cell with said barcode sequence and sample sequence information. In some embodiments, the method further comprises generating a plurality of compartments wherein the cells are sequestered individually in the compartments prior to step (b) or in step (b). In some embodiments, the method further comprises amplifying said barcoded polynucleotide to generate a plurality of amplified barcoded polynucleotides prior to step (c). In some embodiments, the compartments comprise a form of droplet, an emulsion droplet, a liposome, a microwell, a well, a microarray, an open array, a microtiter plate, or a combination thereof. In some embodiments, the sample nucleic acids are selected from the group consisting of a total DNA, a portion of DNA, a total RNA, a portion of RNA and a combination thereof in said cell. In some embodiments, the plurality of barcoded polynucleotides are generated through a reaction selected from a group consisting of ligation, hybridization, strand transfer reaction, transposition, tagmentation, primer extension, reverse transcription, amplification, and a combination thereof. In some embodiments, the sample nucleic acids in the cell are pretreated in situ for reverse transcription, transposition, tagmentation, strand transfer reaction, ligation, hybridization, restriction enzyme digestion, crosslinking, fixation, or a combination thereof before step (b). In some embodiments, the sample sequence with the distinguishable sequence is generated by strand transfer, transposition, tagmentation, random priming, random reverse transcription, random digestion, or a combination thereof. In some embodiments, the sample sequence with the distinguishable sequence is used as a unique molecular identifier for the sample nucleic acid. In some embodiments, at least 80 percent of said sample sequences with the distinguishable sequence comprise a unique sequence different from other sample sequences in said cell. In some embodiments, at least 90 percent of said sample sequences with the distinguishable sequence comprise a unique sequence different from other sample sequences in said cell. In some embodiments, step (d) further comprises using said barcode sequence to identify a cellular origin of the sample nucleic acid and using said sample sequence to determine a uniqueness of the sample nucleic acid from other sample nucleic acids in the cell. In some embodiments, the cells consist essentially of nuclei isolated from the cells.

An embodiment is directed to a method of generating barcoded polynucleotides based on DNA or RNA of a cell comprising (a) providing a sample comprising a plurality of cells, wherein the cell comprises a plurality of sample DNA or sample RNA; (b) generating a plurality of first barcoded polynucleotides from the plurality of sample DNA and a plurality of second barcoded polynucleotides from the plurality of sample RNA of said cell, wherein the first barcoded polynucleotide from sample DNA comprises: a sample sequence from the sample DNA in the cell; a barcode sequence configured to distinguish said sample DNA from other sample DNA in different cells; and a sample DNA specific adapter sequence wherein said adapter sequence comprises the same first barcoded polynucleotide from said sample DNA; wherein the second barcoded polynucleotide from sample RNA comprises a sample sequence from the sample RNA in the cell; a barcode sequence configured to distinguish said sample RNA from other sample RNA in different cells; a sample RNA specific adapter sequence wherein said adapter sequence comprises the same second barcoded polynucleotide from said sample RNA; (c) sequencing said first and the second barcoded polynucleotides to determine the sample sequence and barcode sequence; (d) analyzing the sample DNA and the sample RNA in said cell with said barcode sequence and sample sequence information. In some embodiments, the method further comprises generating a plurality of compartments wherein the cells are sequestered individually in the compartments prior to step (b) or in step (b). In some embodiments, the method further comprises amplifying said first and the second barcoded polynucleotides to generate a plurality of amplified first and second barcoded polynucleotides prior to step (c). In some embodiments, the compartments comprise a form of droplet, an emulsion droplet, a liposome, a microwell, a well, a microarray, an open array, a microtiter plate, or a combination thereof. In some embodiments, the sample DNA is a total DNA, a portion of DNA or an accessible chromatin DNA of said cell. In some embodiments, the sample RNA is a total RNA, a portion of RNA or mRNA of said cell. In some embodiments, the plurality of the first and the second barcoded polynucleotides are generated through a reaction selected from the group consisting of ligation, hybridization, strand transfer reaction, transposition, tagmentation, primer extension, reverse transcription, amplification, and a combination thereof. In some embodiments, the sample DNA in the cell is pretreated in situ for strand transfer reaction, transposition, tagmentation, ligation, hybridization, restriction enzyme digestion, crosslinking, fixation, or a combination thereof before step (b). In some embodiments, the sample RNA in the cell is pretreated in situ for reverse transcription, strand transfer reaction, transposition, tagmentation, ligation, hybridization, restriction enzyme digestion, crosslinking, fixation, or a combination thereof before step (b). In some embodiments, the sample sequence from the first barcoded polynucleotide is a distinguishable sequence from other sample sequences of other sample DNA in said cell. In some embodiments, the sample sequence from the second barcoded polynucleotide is a distinguishable sequence from other sample sequences of other sample RNA in said cell. In some embodiments, the sample sequence with a distinguishable sequence is generated by strand transfer reaction, transposition, tagmentation, random priming, random reverse transcription, random digestion, or a combination thereof. In some embodiments, the sample sequence with a distinguishable sequence is used as a unique molecular identifier for the sample DNA or sample RNA. In some embodiments, at least 80 percent of said sample sequences with a distinguishable sequence comprise a unique sequence different from other sample sequences in said cell. In some embodiments, at least 90 percent of said sample sequences with a distinguishable sequence comprise a unique sequence different from other sample sequences in said cell. In some embodiments,, the barcode sequences are the same between the first and the second barcoded polynucleotides in the cell. In some embodiments, step (d) further comprises using said barcode sequence to identify common cellular origin of the sample DNA or the sample RNA, and using said sample sequences to characterize said sample DNA and said sample RNA in the cell. In some embodiments, the cells consist essentially of nuclei isolated from the cells.

An embodiment is directed to a method of tracking a target's origin by barcode tagging comprising (a) sequestering one or more unique barcode templates with a target in a compartment; (b) amplifying said barcode template and modifying said target wherein the modified target is configured to link a barcode template in the compartment; (c) generating a barcode tagged modified target wherein a plurality of modified targets sharing a same one or more barcode sequences presented in said compartment; and (d) removing the separation between the compartments and collecting the barcode tagged modified targets for sequencing characterization. In some embodiments, the method further comprises identifying a compartment origin of different barcode sequences presented in the same compartment based on a shared compartment content. In some embodiments, the target is selected from the group consisting of a nucleic acid, a protein, a protein complex, a protein and nucleic acid complex, a ligand, a chemical compound, a nucleus, a cell, a microbe, a small molecule, a macromolecule, a particle, a microparticle, and a combination thereof. In some embodiments, the modification for a target is selected from the group consisting of strand transfer reaction, transposition, tagmentation, reverse transcription, amplification, primer extension, restriction enzyme digestion, hybridization, ligation, fragmentation, crosslinking, and a combination thereof. In some embodiments, the target is subject to a treatment and/or a modification before sequestering, wherein the treatment is selected from the group consisting of denaturation, permeabilization, fixation, labeling, antibody conjugation, in situ reaction, and a combination thereof; and wherein the modification is selected from the group consisting of strand transfer reaction, transposition, tagmentation, reverse transcription, amplification, primer extension, restriction enzyme digestion, hybridization, ligation, fragmentation, crosslinking, and a combination thereof. In some embodiments, sequestering compartment is selected from the group consisting of a droplet, an emulsion droplet, a liposome, a microwell, an open array, a microtiter plate, and a combination thereof. In some embodiments, the barcode template comprises a barcode sequence and at least one handle sequence configured to be used as a priming site, a hybridization site or a binding site. In some embodiments, the barcode template is a DNA, a RNA, or a DNA/RNA hybrid and said barcode sequence comprises a range from about 5 bases to about 100 bases. In some embodiments, the method of generating the barcode tagged modified target is through amplification, hybridization, primer extension, ligation, strand transfer reaction, transposition, tagmentation, or a combination thereof. In some embodiments, the target being analyzed is selected from the group consisting of a single cell, a chemical compound, a nucleic acid, a protein, a microbiome, and a combination thereof. An embodiment is directed to methods of amplifiable single cell sequencing to characterize a biological sample at individual cell level. The methods include providing a plurality of cells or nuclei from a sample, providing a plurality of barcode templates, sequestering a cell or a nucleus with more than one different barcode templates in one compartment; amplifying each barcode template into a plurality of copies and amplifying one type or more than one type of cellular content into a plurality of copies, wherein the cellular content comprises nucleic acid sequences naturally or is attached with a nucleic acid sequence artificially, in the sequestered compartment; coupling an amplified barcode template with an amplified cellular content in the compartment; the amplification step and coupling step can happen sequentially or simultaneously; sequencing to determine the barcode sequence in the barcode template and its associated cellular content sequence; classifying the cellular content with the same barcode sequence as one cellular unit. These methods may amplify the cellular contents of a single cell to appear as more than one cellular unit during analysis. The cellular content can be DNA, RNA, protein, lipid, organelle within a cell internally or nucleus or associated with a cell externally. The cell can be eukaryotic and/or prokaryotic. The compartment can be a well, microwell, droplet, microdroplet, hole and other material which is capable to sequester into different reaction units or space. In some embodiments, the barcode templates are oligonucleotides freely in a solution. In some embodiments, the barcode templates are encapsulated in droplets. In some embodiments, the barcoded templates are arranged in a nanoball format. In some embodiments, the barcode templates are immobilized on a carrier clonally (i.e., only one unique barcode sequence with one or multiple copies) or non-clonally (i.e., more than one unique sequence in a single copy or multiple copies). A carrier can be a solid bead or particle, or a dissolvable bead or particle, or a combination thereof.

An embodiment is directed to a method of sequencing a single cell full-length transcriptome comprising providing a plurality of cells from a biological sample; contacting the cells with a reverse transcriptase and an oligo-dT primer to generate first strand cDNA in situ; providing a plurality of transpososomes, each transpososome comprising at least one transposon and one transposase; tagmenting the RNA/cDNA hybrid transcripts randomly across the entire transcripts in situ; providing a plurality of barcode templates and providing amplification reagent; compartmentalizing the cells, the barcode templates, and amplification reagents to generate two or more compartments wherein each compartment comprises a cell, one or more than one barcode templates with different barcode sequences, and amplification reagent; amplifying the barcode template and tagmented RNA/cDNA fragments, attaching said barcode sequence to cDNA fragments or fragments generated from cDNA so that a plurality of barcode attached fragments sharing the same one or more barcode sequences are present in the compartment; collecting the barcode attached fragments; sequencing the barcode and barcoded tagged nucleic acid to characterize the full-length transcriptome profile on a single cell basis. In some embodiments, a nucleus sample replaces a cell sample for the method. In some embodiments, the biological sample is treated with a fixative and/or a permeabilization reagent as a part of procedure.

Although the disclosure has been explained with respect to one or more embodiments, it is to be understood that many other possible modifications and variations can be made without departing from the spirit and scope of the disclosure as herein described.

Further, in general regarding the processes, systems, methods, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the subject matter of the claims.

Moreover, it is to be understood that the above description is intended to be illustrative and not restrictive. Many embodiments and applications other than the examples provided would be apparent to those of skill in the art upon reading the description herein. The scope of the disclosure and described embodiments should be determined, not with reference to the above description, but instead with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the disclosure and the described embodiments are capable of modification and variation and is limited only by the following claims.

Lastly, all defined terms used in the application are intended to be given their broadest reasonable constructions consistent with the definitions provided herein. All undefined terms used in the claims are intended to be given their broadest reasonable constructions consistent with their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides as described herein, and, as such, may be considered in making and practicing the disclosure and embodiments as described herein. Particularly useful techniques for particular embodiments will be discussed in the sections that follow.

The following examples are put forth to provide those of ordinary skill in the art with a complete disclosure and description of how to make and use methods of the present disclosure, and are not intended to limit the scope of the embodiments described herein.

### EXAMPLES

### Example 1: Scalable Method of Single Cell Barcoding

This example describes a scalable method of barcoding the 3′ end of the transcriptome at single-cell resolution that can simultaneously process thousands of cells (FIG. 11).

Human HEK293 cells and mouse NIH-3T3 cells (ATCC, Manassas, VA) were cultured in Dulbecco's Modified Eagle Medium (DMEM) media (Thermo Fisher Scientific, Waltham, MA) with 10% fetal bovine serum (FBS) (Thermo Fisher Scientific, Waltham, MA), supplemented with 1:100 MEM Non-Essential Amino Acids (Thermo Fisher Scientific, Waltham, MA), 1:100 Penicillin/Streptomycin (Thermo Fisher Scientific, Waltham, MA). After reaching 50-80% confluency, cells were harvested with a 1-2-minute treatment of Trypsin-EDTA solution (Thermo Fisher Scientific, Waltham, MA). After dilution with FBS-containing media, cells were washed once with 1×phosphate-buffered solution (PBS) and counted with the Countess-3 Automated Cell Counter system (Thermo Fisher Scientific, Waltham, MA). Approximately 250,000 HEK293 cells and 250,000 mouse NIH-3T3 cells were mixed for this experiment (1:1 ratio) and processed in low-binding 1.5 mL tubes. After centrifugation (300×g for 3 minutes), cells were treated for the purpose of RNA stabilization. Specifically, human and mouse cell mixtures were mildly fixed with a gentle fixative in 100 μL of 1×PBS at room temperature for 45 minutes. Cells were then mildly permeabilized in 100 μL with a mix of non-ionic detergents in PBS at room temperature for 10 minutes. All reactions were conducted in the presence of RNAse and protease inhibitors, and centrifugation steps were conducted at 400×g for 2 minutes in a refrigerated centrifuge. After a cell washing step in 100 μL, aggregates were removed with a filtration step using Flowmi 40 μm cell strainers (Sigma-Aldrich). After a step of cell counting, 50,000 fixed and permeabilized cells were incubated with reserve transcriptase (RT), priming poly-dT oligonucleotide, and dNTP in RT buffer for 30 minutes in a themocycler to synthesize cDNA (RT program: 10 minutes at 50° C., 3 cycles of 12 seconds 8° C., 45 seconds at 15° C., 45 seconds at 20° C., 30 seconds at 30° C., 2 minutes at 42° C., 2.5 minutes at 50° C., and a last step of 5 minutes at 50° C.). After one cell washing step in 100 μL, cDNA molecules inside cells were tagged with transpososome in 20 μL at 37° C. for 20 minutes. After two more cleanup steps and one more step of cell counting, approximately 12,500 cells were mixed with barcode templates and PCR reagents in a volume not larger than 40 μL (adjusted with wash buffer). The cellular solution was mixed with 160 μL of an emulsifying solution (0.2 mL barcoding reaction). In an independent experiment (a different batch of human: mouse cells), approximately 10,000 cells, were mixed with barcode templates and PCR reagents in a total volume of 300 μL, and the aqueous solution was mixed with 700 μL of an emulsifying solution (1.0 mL barcoding reaction). Both aqueous-oil mixtures were aspirated and dispensed for about fifteen minutes under controlled pipetting conditions (50 pipetting iterations) to enable encapsulation of cells and barcoding reagents into droplets. The targeted ratio of number of barcode templates to expected number of droplets was 3 to 1 in order to have approximately 95% of droplets containing at least one barcode template. Emulsions with encapsulated cells and barcoding reagents into droplets were then incubated in a thermocycler for 2 hours for barcode template amplification and cDNA barcoding (PCR program: 5 minutes at 72° C., 30 seconds at 98° C., 20 cycles of 20 seconds at 98° C., 30 seconds at 59° C., 20 seconds at 72° C., 5 cycles of 20 seconds at 98° C., 2 minutes at 40° C., 30 seconds at 72° C., and a final step of 3 minutes at 72° C. The processed emulsions were then incubated with 90 μL (0.2 mL reaction) or 450 μL (1.0 mL reaction) of breaking solution and vortexed for 5 seconds. Oil and cellular debris were separated from soluble molecules by centrifugation at 10,000 rpm for 5 minutes (top layer). Slowly, 125 μL or 625 μL of the aqueous phase, respectively, was transferred into a new tube. After bead cleanup with 130 μL of MagBio magnetic beads (MagBio Genomics), barcoded cDNA fragments were eluted in 40 μL low TE buffer, and indexing and sequencing primers were added to the solution in addition to PCR reagents to generate an Illumina compatible library (PCR program: 30 seconds at 98° C., 8 cycles of 20 seconds at 98° C., 30 seconds at 62° C., and 40 seconds at 72° C. with a final cycle of 2 minutes at 72° C.). After a new clean-up step with MagBio magnetic beads (0.9×), the final library was quantified and sized using a 4200 TapeStation system and high sensitivity D1000 reagents (Agilent, La Jolla, CA). The average size and concentration of the library was 414 base pairs (bp) and 10 mM, respectively.

The library was sequenced in a single end run on a NextSeq system (Illumina, San Diego, CA). Sequencing configuration: Read 1, single-end read 90 cycles (transcript); Index 1 (i7), 8 cycles (sample index); index 2 (i5), 20 cycles (barcode templates). Sequencing depth: the total number of reads was 103,412,571 for the 0.2 mL reaction (91.2% reaps mapped to genome) and 103,298,991 for the 1.0 mL reaction (84.7% reads mapped to genome).

After bcl-to-fastq conversion and demultiplexing of the sequencing data, barcode templates were error corrected, adapter sequences were trimmed, and duplicate reads were removed. For barcode template grouping, the plurality of barcode templates capturing the content from the same cell was estimated and integrated. The resulting reads were mapped to a mixture of the reference human and mouse genomes (hg38 and Mm10) using Cell Ranger v5.0.1 software (10×Genomics), and cells were distinguished from background using a barcode ranked plot based on the same software: 10,099 estimated cells in the 0.2 mL experiment (5,149 human cells and 5,298 mouse cells; fraction reads in cells, 80.3%; 10,240 mean reads per cell; 2,337 median human genes per cell; 2,028 median mouse genes per cell; 28,203 total human genes; 20,339 total mouse genes) and 6,715 estimated cells in the 1.0 mL experiment (4,035 human cells and 2,699 mouse cells; fraction reads in cells, 68.1%; 15,383 mean reads per cell; 1,181 median human genes per cell; 1,933 median mouse genes per cell; 27,789 total human genes; 19,755 total mouse genes).

To verify the single-cell behaviors in both experiments (FIG. 11), Cell Ranger-generated expression outputs were processed with Seurat v4.0 software (developed by the Satija laboratory at the New York Genome Center, New York University). Visualization of cells based on the number of human and mouse reads shows that cells mostly distribute along the axes, in agreement with distinctively single-cell human or mouse properties. Only a relatively small fraction of cells, estimated as 3.91% for the 0.2 mL reaction (1101) and 0.48% for the 1.0 mL reaction (1102), could be attributed to collisions between human and mouse cells in the same droplet. The collision rate was therefore estimated as 7.48% for the 0.2 mL experiment (10,099 cells) and 0.95% for the 1.0 mL experiment (6,715 cells). This difference in collision rates supports that cell collisions depend on barcoding reaction volume. The scalability of this reaction could be used for the purpose of diminishing collision rates or to increase throughput to up to 62,500 cells in a 1 mL barcoding reaction.

To further validate the single-cell behavior of the barcoding reactions, expression of a representative human gene (1103 and 1104) or mouse gene (1105 and 1106) on t-SNE plots in the 0.2 mL experiment were highlighted. (FIG. 11). The expression patterns of both genes were, overall, mutually exclusive across the cell population. Importantly, these patterns were virtually indistinguishable whether the plurality of barcode templates was processed without grouping (1103 and 1105) or whether barcodes templates were first computationally grouped to infer cells (1104 and 1106). This observation suggests that the process of barcode grouping reconstructs cellular content without inferring a significant number of artifactual human: mouse cells. Further in agreement, the estimated fraction of co-encapsulated human: mouse cells based on the UMAP plot is relatively similar whether skipping the process of barcoding grouping or performing this step, 4.55% and 3.38% respectively (1103 vs. 1104 or 1105 vs. 1106).

### Example 2: 3′ Single Cell RNA-Seq Analysis of a Sample With a Plurality of Human Cells (PBMC) Extracted From Peripheral Human Blood

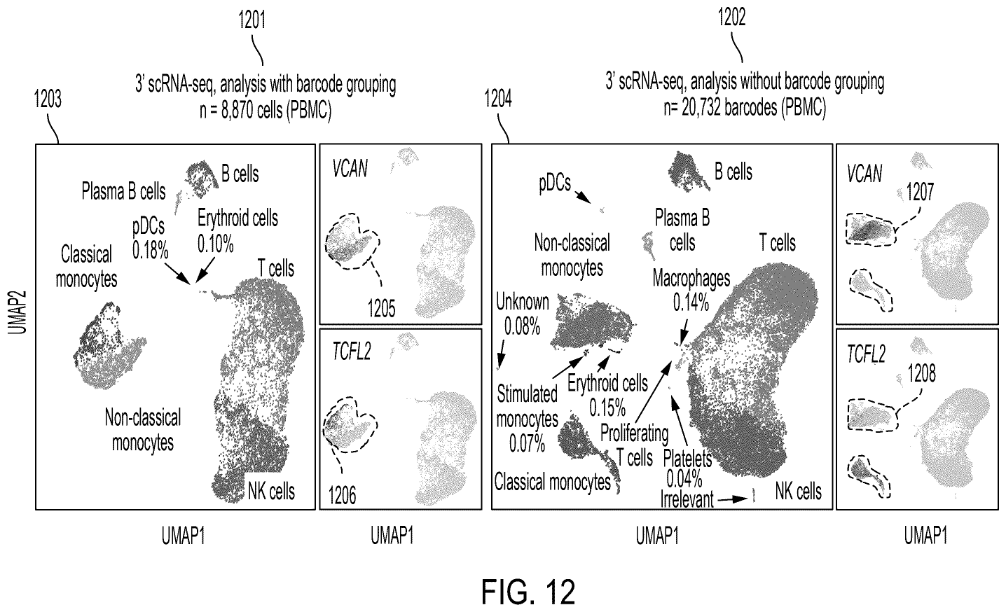

This example describes a method of barcoding the 3′ end of the transcriptome at single-cell resolution that can identify a plurality of cell types in a sample of human PBMC derived from peripheral blood (FIG. 12).

Approximately 10 million cryopreserved PBMC (AllCells, Alameda, CA) were gently thawed and 1M cells were processed as described in Example 1 after the step of cell harvesting. Libraries were then generated and sequenced also as described in Example 1 (0.2 mL size reaction). Sequencing depth: 120,326,303 reads.

As described in Example 1, cells were distinguished from background using a barcode ranked plot: 8,870 estimated cells after barcode template grouping (fraction reads in cells, 87.6%; 8,063 mean reads per cell; 820 median genes per cell), or 20,723 cell-associated barcodes when skipping the process of barcode template grouping (fraction reads in cells, 85.3%; 4,827 mean reads per cell; 612 median genes per cell).

FIG. 12 shows UMAP visualization of 3′ single cell RNA-seq data. The figure illustrates two analysis methods: one method is based on grouping the plurality of barcodes with similar compartment content to estimate cells (1201); and the other method is based on individual barcode templates without undergoing this process of barcode grouping (1202). The figure highlights the identification of the expected PBMC types after barcode grouping (1203: B cells, plasma B cells, classical monocytes, non-classical monocytes, T cells, NK cells and rare cell populations such as plasmacytoid dendritic cells or pDCs cells, 0.18%, and erythroid cells, 0.1%). Notably, the analysis with non-grouped barcodes (1204) shows a higher resolution than with grouped barcodes, with the identification of additional rare cell populations using the same data (including proliferating T cells, macrophages, stimulated monocytes, and platelets; the latter representing no more than 0.04% of the total number of cell-associated barcodes detected). Further in support of a higher resolution for the analysis method based on un-grouped barcodes, the two major monocytic populations (classical and non-classical cells) are more clearly separated with un-grouped barcodes, which can be better observed when highlighting the expression of two cell-type-specific gene markers (VCAN and TCFL2) in UMAP plots generated with both analysis methods. VCAN is a maker for non-classical monocytes (1205 with barcode merging and 1207 without barcode merging), and TCFL2 is a marker for classical monocytes (1206 with barcode merging and 1208 without barcode merging).

### Example 3: Full-Length Single Cell RNA-Seq Analysis of Human Jurkat Cells

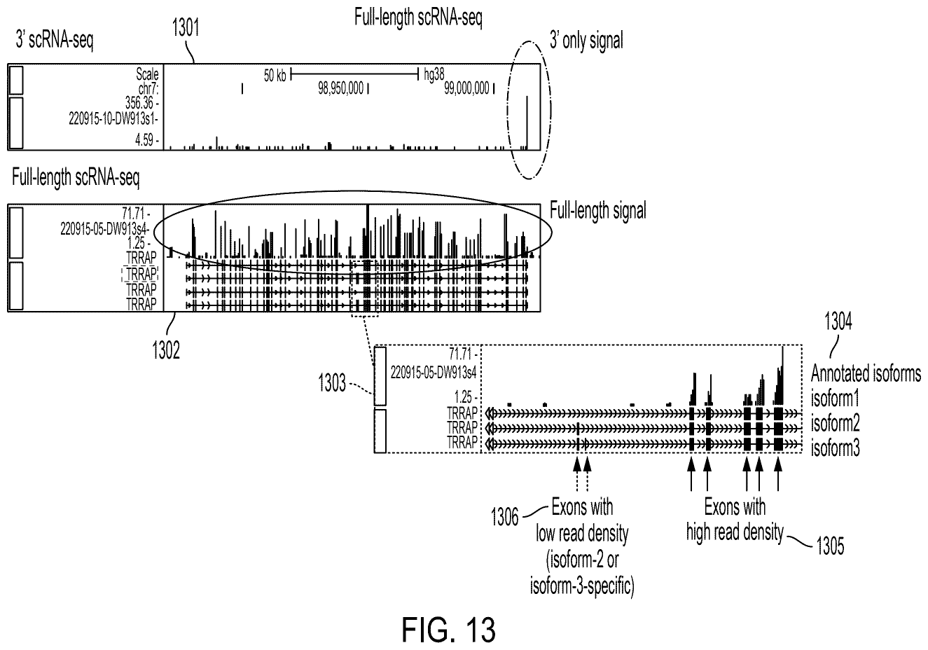

This example describes a method of barcoding full-length transcripts at single-cell resolution (FIG. 13).

Human Jurkat cells (ATCC, Manassas, VA) were cultured in DMEM media (Thermo Fisher Scientific, Waltham, MA) with 10% FBS (Thermo Fisher Scientific, Waltham, MA), supplemented with 1:100 MEM Non-Essential Amino Acids (Thermo Fisher Scientific, Waltham, MA), 1:100 Penicillin/Streptomycin (Thermo Fisher Scientific, Waltham, MA). After reaching a confluency of half a million cells per mL, cells were harvested by centrifugation, and washed in 1×PBS. Approximately, 0.5 million Jurkat cells were processed as described in Example 1. Libraries were then generated and sequenced also as described in Example 1 (0.2 mL size reaction). The main difference is the addition of random hexamers to the RT reaction as priming oligos, and the use of transpososome activities with two different assembled sequences (not only one, Tn5A) during the step of cDNA tagmentation, Tn5A and Tn5B. Sequencing depth: 46,281,274 reads.

As described in Example 1, cells were distinguished from background using a barcode ranked plot: 1,526 estimated cells after barcode template grouping (fraction reads in cells, 73.8%; 30,328 mean reads per cell; 1,624 median genes per cell). Sequencing reads were also processed as an aggregate from all cells (so-called ‘pseudo-bulk’ analysis) and visualized as tracks of read density using the University of California at Santa Cruz (UCSC) Genome Browser.

FIG. 13 shows UCSC Browser tracks of pseudo-bulk read density along a representative gene using the 3′ (1301) and full-length (1302) methods of cDNA priming and single/dual tagmentation (Tn5A/Tn5A & Tn5B). This figure shows read coverage mostly concentrated around the 3′ end of the annotated gene when processing the library with the 3′ scRNA-seq method (1301), as opposed to the observation of read coverage across most of the annotated exons when processing the library with the full-length scRNA-seq method (1302). The selected gene has at least three annotated isoforms (1303): isoform 1-3 (1304). While most exons are shared by all three isoforms (1305), which were densely covered by reads, a few isoform-specific exons were virtually not covered by any read, suggesting the low or no expression of the isoforms containing these exons, isoforms 2 and 3 (1306).

### Example 4: Microbial Single Cell Genomic Analysis of a Mock Mixture of Five Different Bacterial Species

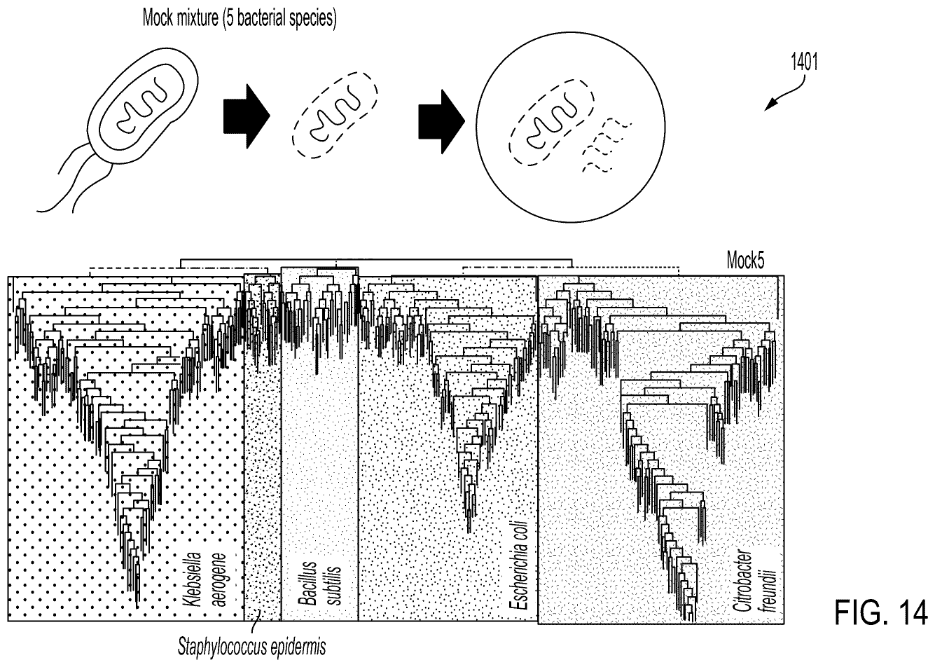

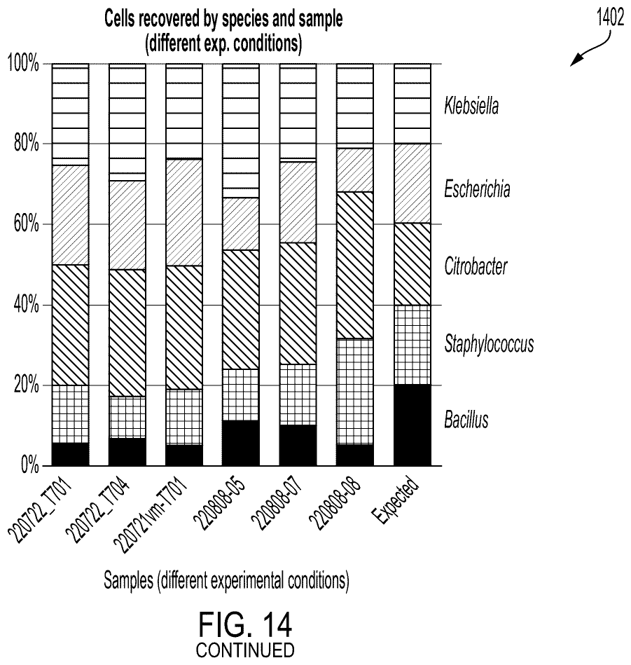

This example describes a method of barcoding DNA fragments underlying random genomic regions at single-cell resolution for the purpose of taxonomy (FIG. 14).

Five reference bacterial species (purchased from ATCC) were cultured separately in LB broth at saturation and mixed at a 1:1:1:1:1 ratio prior to cell permeabilization (Mock 5, three gram-negative cells and two gram-positive cells): Escherichia coli (−), Bacillus subtilis (+), Citrobacter freundii (−), Klebsiella aerogenes (−), and Staphylococcus epidermidis (+). After mixing, cells were washed in 1×PBS (spins at 600×g for 5 minutes at room temperature, swing bucket rotor). 10M cells (absorbance quantification at OD600) were mildly fixed for 45 minutes at room temperature and washed two times with 1×PBS prior to permeabilization. On ice, cells were permeabilized with 0.04% Tween-20 for 3 minutes. After cell centrifugation (600×g for 5 minutes), cells were further permeabilized with 4 μg of lysostaphin (Sigma-Aldrich) and 10 μg of lysozyme (Sigma-Aldrich) for 30 minutes at 37° C. Before a new centrifugation, cold 1×PBS was added and cells were pelleted at 600×g for 5 minutes. After two more washing steps in cold PBS, the tagmentation reaction was conducted with a mix of Tn5A and Tn5B transpososomes at 37° C. for 1 hour. Between 30,000-250,000 cells were used for cell encapsulation and processed as described in Example 1.

Using the plurality of read sequences and their barcodes without the aid of a reference genome, FIG. 14 shows that unsupervised hierarchical clustering of read sequences segregates the plurality of barcodes by bacterial origin (1401). Briefly, the annotations of the five bacterial genomes were leveraged to distinguish barcodes based on the origin of their associated reads (genomic content). Specifically (1401), barcodes containing mainly Klebsiella aerogenes reads were clustered together; barcodes containing primarily Staphylococcus epidermidis reads were clustered together; barcodes containing primarily Bacillus subtilis reads were clustered together; barcodes containing primarily Escherichia coli reads were clustered together; and barcodes containing primarily Citrobacter freundii reads were clustered together. As observed in the figure, content-based barcode clustering segregated barcodes by species in support of the method of barcoding as described herein that can capture taxonomic information at single-cell resolution. Furthermore, different cell permeabilization and encapsulation conditions consistently show that the abundance of each species can be estimated to a certain degree, except for Bacillus subtilis and sometimes also Staphylococcus epidermidis, as expected based on their gram-positive identity (1402). Overall, these results suggest that bacterial cells can be processed with the barcoding method as described herein for the purpose of bacterial taxonomic identification and that these cells can also be quantified if permeabilized efficiently.

### REFERENCES

Adey A. et al. 2010. Genome Biol. 11, R119.
Amini S. et al. 2014. Nature Genetics, 46(12):1343-1349.
Au, T. et al. 2004. EMBO J., 23:3408-3420.
Buenrostro J. D. et al. 2013. Nature Methods, 10(12): 1213-1218.
Buenrostro, J. D. et al. 2015. Nature, 523: 486-490.
Burton B. M. and Baker T. A. 2003. Chemistry & Biology 10: 463-472.
Caruccio N. 2011. Methods Mol. Biol. 733: 241-255.
Kavanagh I, Kiiskinen L. L. and Haakana H. 2013. Unite State Patent Application Publication U.S.2013/0023423.
Kurihara K. et al. 2011. Nat. Chem. 3: 775-781.
Laouini A. et al. 2012. Colloid Sci. Biotechnol. 1: 147-168.
Mizuuchi M., Baker T. A. and Mizuuchi K. 1992. Cell 70, 303-311.
Savilahti H., P. A. Rice, and K. MiZuuchi. 1995. EMBO J. 14:4893-4903.
Stoeckius M., et al. 2017. Nature Methods 14: 865-868.
Surette M., Buch S. J. and Chaconas G. 1987. Cell 70: 303-311.
Reznikoff W. S. 2008. Annual Review of Genetics 42(1): 269-286.

What is claimed:

1. A method of single cell sequencing to characterize a biological sample at an individual cell level, the method comprising:
a) sequestering a plurality of cells or a plurality of nuclei into compartments, wherein each cell or nucleus is sequestered into a separate compartment with a plurality of barcode templates, wherein each barcode template comprises a barcode sequence, and wherein at least some compartments comprise more than one population of barcode templates, each population of barcode templates having a unique barcode sequence different from that of other populations of barcode templates;
b) amplifying at least one type of cellular content in each cell or nucleus into a plurality of copies and fragmenting the cellular content in each compartment into a plurality of fragments;
c) attaching a barcode template to each fragment;
d) collecting the barcode template attached fragments; and
e) sequencing the barcode attached fragments and classifying fragments with a same barcode sequence as belonging to a same cellular unit.

2. A method of single cell sequencing to characterize a biological sample at an individual cell level, the method comprising:
a) sequestering a plurality of cells or a plurality of nuclei and a plurality of barcode templates into compartments, wherein each cell or nucleus is sequestered into a separate compartment with at least one barcode template comprising a barcode sequence, and wherein at least some compartments comprise at least two different barcode templates, each different barcode template having a different barcode sequence;
b) amplifying at least one type of cellular content in each cell or nucleus into a plurality of copies and fragmenting the cellular content in each compartment into fragments, and amplifying the at least one barcode template in each compartment;
c) attaching a barcode template to each fragment;
d) collecting the barcode template attached fragments; and
e) sequencing the barcode attached fragments and classifying fragments with a same barcode sequence as belonging to a same cellular unit.

3. The method of claim 1, wherein each barcode template
a) is a nucleotide sequence, capable of functioning as a unique identifier;
b) exists freely in solution; and/or
c) is immobilized on a carrier selected from the group consisting of a solid bead or particle, a dissolvable bead or particle, or a combination thereof.

4. The method of claim 1, wherein the type of cellular content is RNA, DNA, RNA/DNA hybrid, protein, metabolite, ligand, chemical compound, drug, macromolecule, or a combination thereof.

5. The method of claim 1, wherein the fragment is directly or indirectly attached to the barcode template.

6. The method of claim 1, wherein the fragment is attached to a linker oligo, or an adapter, wherein the linker oligo or the adapter is attached to the barcode template.

7. The method of claim 1, wherein the cellular content is endogenous or exogenous.

8. The method of claim 1, wherein the compartment comprises a cell or a nucleus without further compartmentation; a tube or microtube; a well or microwell; a plate; a well in a multi-well plate; a slide; a spot on a slide; a droplet; a tubing; a channel; a bottle; a chamber; or a flow-cell.

9. The method of claim 1, further comprising identifying barcode sequences attached to cellular content originating from the same cell or nucleus, and merging cellular units corresponding to barcode sequences identified as attached to cellular content originating from the same cell or nucleus.

10. A method of single cell transcriptome sequencing, the method comprising:
a) generating cDNA from cellular or nuclear RNA of a cell or nucleus in a plurality of cells or nuclei;
b) tagmenting the generated cDNA randomly across an entire length of the cDNA in each of the cells or nuclei using a plurality of transpososomes, to form a plurality of tagmented cDNA fragments, wherein each transpososome comprises at least one transposon and one transposase;
c) sequestering the plurality of cells or nuclei into compartments, wherein each cell or nucleus is sequestered into a separate compartment with a plurality of barcode templates, wherein each barcode template comprises a barcode sequence;
d) attaching a barcode template to each tagmented cDNA fragment in the compartment;
e) collecting the barcode attached cDNA fragments;
f) sequencing the barcode and barcode attached cDNA fragments to characterize a transcriptome profile of each cell or nucleus on a single cell basis.

11. A method of single cell transcriptome sequencing, the method comprising:
a) generating cDNA from cellular or nuclear RNA from a cell or nucleus in a plurality of cells or nuclei;
b) tagmenting the generated cDNA randomly across an entire length of the cDNA in each of the cells or nuclei using a plurality of transpososomes, to form a plurality of tagmented cDNA fragments, wherein each transpososome comprises at least one transposon and one transposase;
c) sequestering the cells or nuclei and a plurality of barcode templates, wherein each cell or nucleus is sequestered into a separate compartment with at least one barcode template;
d) attaching a barcode template to each tagmented cDNA fragment;
e) collecting the barcode attached cDNA fragments;
f) sequencing the barcode and barcode attached cDNA fragments to characterize the transcriptome profile of each cell on a single cell basis.

12. The method of claim 10, wherein the plurality of barcode templates in each compartment comprises at least two populations of barcode templates, wherein each population of barcode templates has a different barcode sequence.

13. The method of claim 10, wherein the attaching results in at least two populations of cDNA fragments each attached to a different population of barcode templates.

14. The method of claim 10, wherein the at least one barcode template is at least two different barcode templates, each having a different barcode sequence.

15. The method of claim 10, wherein the generated cDNA comprises
a) first strand cDNA and forms a DNA/RNA hybrid with the cellular or nuclear RNA;
b) first and second stranded cDNA, and forms double stranded DNA; or
c) transcripts comprising both a 3′ end and a 5′ end of the cellular or nuclear RNA or wherein the transcriptome profile comprises both a 3′ end and a 5′ end of the cellular or nuclear RNA.

16. The method of claim 10, wherein attaching the barcode template to the tagmented cDNA fragment comprises amplifying the barcode templates and/or amplifying the tagmented cDNA fragments.

17. The method of claim 16, wherein amplifying the barcode templates and the amplifying the tagmented cDNA fragments occurs separately or simultaneously.

18. The method of claim 10, wherein the at least one barcode template in each compartment is a single barcode template

19. The method of claim 10, wherein the plurality of barcode templates in each compartment is a plurality of copies of a same barcode template.

20. The method of claim 10, wherein each barcode template exists freely in solution or is immobilized on a carrier.
